(19) United States
(12) Patent Application Publication
(10) Pub. No.: US 2009/0304825 A1
(43) Pub. Date: Dec. 10, 2009

(54) ESTROGENIC EXTRACTS FOR USE IN TREATING VAGINAL AND VULVAR ATROPHY

(75) Inventor: Isaac Cohen, Piedmont, CA (US)

Correspondence Address: WILSON, SONSINI, GOODRICH & ROSATI, 650 PAGE MILL ROAD, PALO ALTO, CA, 94304-1050, US

(73) Assignee: Bionovo, Inc., Emeryville, CA (US)

(21) Appl. No.: 12/436,020

(22) Filed: May 5, 2009

Related U.S. Application Data
(60) Provisional application No. 61/050,925, filed on May 6, 2008.

(51) Int. Cl. A61K 31/35; A61K 36/00; C12N 5/02

(52) U.S. Cl. 424/725; 514/456; 435/375

(57) ABSTRACT

Estrogenic extracts of Astragalus membranaceus, Astragalus mongholicus, Anemarrhenae asphodeloides and Achyranthes bidentata are provided. Also provided are methods of using said extracts to achieve an estrogenic effect, especially in a human, e.g. a female human. In some embodiments, the methods include treatment of climacteric symptoms. In some embodiments, the methods include treatment or prevention of osteoporosis.

### CROSS-REFERENCE AND PRIORITY CLAIM

This application claims priority under 35 U.S.C. §119(e) from U.S. provisional patent application No. 61/050,925, filed May 6, 2008, which is incorporated herein by reference in its entirety.

### FIELD OF THE INVENTION

The present invention relates to plant extract compositions and more particularly to compositions comprising extracts of plant species belonging to the species Astragalus membranaceus, Astragalus mongholicus, Anemarrhenae asphodeloides and Achyranthes bidentata. The invention further relates to methods of using and methods of making such plant extract compositions.

### BACKGROUND OF THE INVENTION

The onset of menopausal vaginal atrophy is associated with the decline of circulating estrogens. From perimenopause to postmenopause, serum estrogen levels have been shown to drop from approximately 400 pg/ml to less than 10 pg/ml. Estrogen replacement treatment has been used successfully for the treatment of vaginal dryness. It is hypothesized that the effect is accomplished via agonistic stimulation of the estrogen receptors in the vulva and vaginal canal.

### SUMMARY OF THE INVENTION

The present inventor has identified a need for estrogenic compositions useful for the treatment of one or more disease states associated with the estrogen receptor. The inventor has also identified a need for estrogenic compositions that do not increase the risk or likelihood that a patient administered the compositions will suffer from another disease state associated with an estrogen receptor. The inventor has likewise recognized a need for an estrogenic composition that will reduce the risk of one or more estrogen receptor mediated disease states while, at the same time, treating another estrogen receptor mediated disease state. The inventor has also identified a need for estrogenic compositions that are readily obtained from natural sources, as well as a need for methods of making and using such estrogenic compositions. The disclosure herein meets such needs and provides related advantages as well.

Thus, in some embodiments, the invention provides a composition, comprising a therapeutically effective amount of an extract of Astragalus membranaceus, Astragalus mongholicus, Anemarrhenae asphodeloides and Achyranthes bidentata, wherein the extract comprises at least one phytochemical. In some embodiments, the composition comprises one or more pharmaceutically acceptable excipients and a therapeutically effective amount of an extract of Astragalus membranaceus, Astragalus mongholicus, Anemarrhenae asphodeloides and Achyranthes bidentata. In some embodiments, the composition consists essentially of one or more pharmaceutically acceptable excipients and a therapeutically effective amount of an extract of Astragalus membranaceus, Astragalus mongholicus, Anemarrhenae asphodeloides and Achyranthes bidentata. In some embodiments, the composition consists of one or more pharmaceutically acceptable excipients and a therapeutically effective amount of an extract of Astragalus membranaceus, Astragalus mongholicus, Anemarrhenae asphodeloides and Achyranthes bidentata. In some embodiments, the therapeutically effective amount of an extract of Astragalus membranaceus, Astragalus mongholicus, Anemarrhenae asphodeloides and Achyranthes bidentata is about 1 gram dry weight to about 100 grams dry weight of the extract per day. In some embodiments, the therapeutically effective amount of an extract of Astragalus membranaceus, Astragalus mongholicus, Anemarrhenae asphodeloides and Achyranthes bidentata is about 10 grams dry weight to about 100 grams dry weight of the extract per day. In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone. In some embodiments, the purified extract of phytochemicals is obtained by performing a first extraction and a second extraction. In some embodiments, the first extraction comprises the steps of: (a) dispersing Astragalus membranaceus, Astragalus mongholicus, Anemarrhenae asphodeloides and Achyranthes bidentata in a first solvent to form a first mixture; (b) heating the first mixture; and (c) removing the first solvent to obtain a first extract. In some embodiments, the second extraction comprises the steps of: (a) mixing the first extract with a second solvent to produce a second mixture; (b) allowing the second mixture to partition into an aqueous layer and a non-aqueous layer; (c) collecting the non-aqueous layer; (d) removing any non-active compounds; and (e) removing the second solvent to obtain a purified extract of phytochemicals. In some embodiments, the first solvent is a solution of ethanol and water. In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. In some embodiments, the first mixture is heated for about 2 hours to about 6 hours. In some embodiments, the first mixture is heated for about for about 4 hours. In some embodiments, the first solvent is removed from the first extract by evaporation. In some embodiments, the evaporation takes place at a temperature from about 40° C. to about 60° C. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation. In some embodiments, the composition further comprises at least one additional ingredient selected from the group consisting of: active pharmaceutical ingredients; enhancers; excipients; and agents used to adjust the pH, buffer the composition, prevent degradation and improve appearance, odor or taste.

Some embodiments described herein provide a process of isolating a purified extract of phytochemicals from the plant species Astragalus membranaceus, Astragalus mongholicus, Anemarrhenae asphodeloides and Achyranthes bidentata. In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone. In some embodiments, the purified extract of phytochemicals is obtained by performing a first extraction and a second extraction. In some embodiments, the first extraction comprises the steps of: (a) dispersing Astragalus membranaceus, Astragalus mongholicus, Anemarrhenae asphodeloides and Achyranthes bidentata in a first solvent to form a first mixture; (b) heating the first mixture; and (c) removing the first solvent to obtain a first extract. In some embodiments, the second extraction comprises the steps of: (a) mixing the first extract with a second solvent to produce a second mixture; (b) allowing the second mixture to partition into an aqueous layer and a non-aqueous layer; (c) collecting the non-aqueous layer; (d) removing any non-active compounds; and (e) removing the second solvent to obtain a purified extract of phytochemicals. In some embodiments, the first solvent is a solution of ethanol and water. In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. In some embodiments, the first mixture is heated for about 2 hours to about 6 hours. In some embodiments, the first mixture is heated for about for about 4 hours. In some embodiments, the first solvent is removed from the first extract by evaporation. In some embodiments, the evaporation takes place at a temperature from about 40° C. to about 60° C. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation. In some embodiments, the composition further comprises at least one additional ingredient selected from the group consisting of: active pharmaceutical ingredients; enhancers; excipients; and agents used to adjust the pH, buffer the composition, prevent degradation and improve appearance, odor or taste.

Some embodiments described herein provide a method of treating a subject with a disorder which may be treatable with a composition comprising a therapeutically effective amount of a purified extract of phytochemicals from Astragalus membranaceus, Astragalus mongholicus, Anemarrhenae asphodeloides and Achyranthes bidentata. In some embodiments, the disorder is vaginal atrophy, vulvar atrophy, or a symptom of one or both thereof (e.g. vaginal dryness, irritation, itching, bleeding and/or dyspareunia increase). In some embodiments, the disorder is one or more climacteric symptoms (i.e. symptoms associated with perimenopause, menopause or post-menopause). In some embodiments, the composition comprises one or more pharmaceutically acceptable excipients and a therapeutically effective amount of an extract of Astragalus membranaceus, Astragalus mongholicus, Anemarrhenae asphodeloides and Achyranthes bidentata. In some embodiments, the composition consists essentially of one or more pharmaceutically acceptable excipients and a therapeutically effective amount of an extract of Astragalus membranaceus, Astragalus mongholicus, Anemarrhenae asphodeloides and Achyranthes bidentata. In some embodiments, the composition consists of one or more pharmaceutically acceptable excipients and a therapeutically effective amount of an extract of Astragalus membranaceus, Astragalus mongholicus, Anemarrhenae asphodeloides and Achyranthes bidentata. In some embodiments, the therapeutically effective amount of an extract of Astragalus membranaceus, Astragalus mongholicus, Anemarrhenae asphodeloides and Achyranthes bidentata is about 1 gram dry weight to about 100 grams dry weight of the extract per day. In some embodiments, the therapeutically effective amount of an extract of Astragalus membranaceus, Astragalus mongholicus, Anemarrhenae asphodeloides and Achyranthes bidentata is about 10 grams dry weight to about 100 grams dry weight of the extract per day. In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone. In some embodiments, the purified extract of phytochemicals is obtained by performing a first extraction and a second extraction. In some embodiments, the first extraction comprises the steps of: (a) dispersing Astragalus membranaceus, Astragalus mongholicus, Anemarrhenae asphodeloides and Achyranthes bidentata in a first solvent to form a first mixture; (b) heating the first mixture; and (c) removing the first solvent to obtain a first extract. In some embodiments, the second extraction comprises the steps of: (a) mixing the first extract with a second solvent to produce a second mixture; (b) allowing the second mixture to partition into an aqueous layer and a non-aqueous layer; (c) collecting the non-aqueous layer; (d) removing any non-active compounds; and (e) removing the second solvent to obtain a purified extract of phytochemicals. In some embodiments, the first solvent is a solution of ethanol and water. In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. In some embodiments, the first mixture is heated for about 2 hours to about 6 hours. In some embodiments, the first mixture is heated for about for about 4 hours. In some embodiments, the first solvent is removed from the first extract by evaporation. In some embodiments, the evaporation takes place at a temperature from about 40° C. to about 60° C. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation. In some embodiments, the composition further comprises at least one additional ingredient selected from the group consisting of: active pharmaceutical ingredients; enhancers; excipients; and agents used to adjust the pH, buffer the composition, prevent degradation and improve appearance, odor or taste.

Some embodiments described herein provide a method of activating a gene under control of an estrogen response element, comprising administering to a cell having an estrogen response element operatively linked to the gene and an estrogen receptor an amount of a composition, wherein the composition comprises a purified extract of phytochemicals from Astragalus membranaceus, Astragalus mongholicus, Anemarrhenae asphodeloides and Achyranthes bidentata, and wherein the purified extract of phytochemicals is in an amount sufficient to activate the gene. In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone. In some embodiments, said cell is in vitro. In some embodiments, said cell is in vivo. In some embodiments, said cell is in an ERα+ breast tissue. In some embodiments, said cell is in an ERβ+ breast tissue. In some embodiments, said cell is in an ERα/ERβ+ breast tissue. In some embodiments, said estrogen response element is expressed in a transformed cell. In some embodiments, both the estrogen response element and the estrogen receptor are expressed in the cell. In some embodiments, said estrogen response element is heterologously expressed in the cell. In some embodiments, both the estrogen response element and the estrogen receptor are heterologously expressed in the cell. In some embodiments, said cell is selected from the group consisting of a U937, a U2OS, a MDA-MB-435 and a MCF-7 cell transformed with an ERE-controlled gene. In some embodiments, the cell expresses ERα. In some embodiments, the cell expresses ERβ. In some embodiments, the ERE-controlled gene is ERE-tk-Luc.

Some embodiments described herein provide a process of repressing expression of a TNF RE-controlled gene, comprising administering to a cell comprising a gene under control of a TNF response element and an estrogen receptor an amount of a composition, wherein the composition comprises a purified extract of phytochemicals from Astragalus membranaceus, Astragalus mongholicus, Anemarrhenae asphodeloides and Achyranthes bidentata and wherein the purified extract of phytochemicals is in an amount sufficient to repress the TNF RE-controlled gene. In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone. In some embodiments, the TNF RE-controlled gene is TNF-α. In some embodiments, the TNF RE-controlled gene is TNF RE-Luc. In some embodiments, said cell is in vitro. In some embodiments, said cell is in vivo. In some embodiments, said cell is in an ER+ breast tissue. In some embodiments, said cell is in an ERα+ breast tissue. In some embodiments, said cell is in an ERβ+ breast tissue. In some embodiments, said TNF response element is endogenously expressed in the cell. In some embodiments, both the TNF response element and the estrogen receptor are endogenously expressed in the cell. In some embodiments, said TNF response element is heterologously expressed in the cell. In some embodiments, both the TNF response element and the estrogen receptor are heterologously expressed in the cell. In some embodiments, said cell contains an estrogen receptor gene, is transformed with an TNF response element-controlled gene and is selected from the group consisting of a U937, a U2OS, a MDA-MB-435 and a MCF-7 cell. In some embodiments, the estrogen receptor gene is a gene expressing ERα. In some embodiments, the estrogen receptor gene is a gene expressing ERβ.

### Incorporation by Reference

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized and the accompanying drawings of which:

FIGS. 1A-1C. U2OS osteosarcoma cells were cotransfected with ERE-tk-Luc and expression vectors for human ERα or ERβ. The herbal extracts and their active chemical ingredients in VG101 produced a dose-dependent activation of ERE-tk-Luc with ERβ, but not with ERα. FIG. 1A: the effects of Rhizomae Anemarrhenae asphodeloides Bunge (Liliaceae); FIG. 1B: Radix Astragali (Leguminosae/Fabaceae); FIG. 1C: Radix Achyranthes bidentata B1. (Amarathaceae).

FIGS. 2A-2D. The herbal extracts and their active chemical ingredients caused activation equivalent to 10 nM estradiol (E₂). The ER antagonists raloxifene (Ral) and tamoxifen (Tam) blocked the activation by: the effects of Rhizomae Anemarrhenae asphodeloides Bunge (Liliaceae)(FIG. 2A); Radix Astragali (Leguminosae/Fabaceae)(FIG. 2B), Radix Achyranthes bidentata B1. (Amarathaceae)(FIG. 2C), Nyasol (FIG. 2D).

FIGS. 3A-3E. Expression of TNFα gene mRNA in tetracycline-inducible ERα or ERβ U2OS cells treated with estrodiol (E₂) or herbs. FIG. 3A: TNFα produced a large increase in TNFα mRNA, which was inhibited by E₂ in both the U2OS-ERα; FIG. 3B: U2OS-ERβ; FIG. 3C: dose response of Rhizomae Anemarrhenae asphodeloides Bunge (Liliaceae); FIG. 3D: Radix Astragali (Leguminosae/Fabaceae); FIG. 3E: Radix Achyranthes bidentata B1. (Amarathaceae) on U2OS-ERα and U2OS-ERβ TNFα repression. The herbal extracts and their active chemical ingredients in VG101 do not stimulate MCF-7 cell tumor formation or uterine growth in mouse xenograft models.

FIGS. 4A-4C. Herbal extract were tested with CyQuant Cell Proliferation Assay in BT474, ER+ breast cancer cells and MDA-MB-468, ER− breast cancer cells. The cells were treated for 48 hours with the herbal extracts. FIG. 4A: the effects of Rhizomae Anemarrhenae asphodeloides Bunge (Liliaceae); FIG. 4B: Radix Astragali (Leguminosae/Fabaceae); FIG. 4C: Radix Achyranthes bidentata (Amarathaceae).

### DETAILED DESCRIPTION OF THE INVENTION

In some embodiments, the invention comprises a composition, comprising a therapeutically effective amount of an extract of Astragalus membranaceus, Astragalus mongholicus, Anemarrhenae asphodeloides and Achyranthes bidentata.

In some embodiments, the phytochemical is selected from the group consisting of: calycosin, nyasol and tetracyclic isoflavone. In some embodiments, the extract comprises at least two of: calycosin, nyasol and tetracyclic isoflavone.

In some embodiments, the extract comprises calycosin, nyasol and tetracyclic isoflavone.

In some embodiments, the purified extract of phytochemicals is obtained by performing a first extraction and a second extraction.

In some embodiments, the first extraction comprises the steps of: dispersing Astragalus membranaceus, Astragalus mongholicus, Anemarrhenae asphodeloides and Achyranthes bidentata, in a first solvent to form a first mixture; heating the first mixture; and removing the first solvent to obtain a first extract. In some embodiments, the first solvent is a solution of ethanol and water. In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. In some embodiments, the first mixture is heated for about 2 hours to about 6 hours. In some embodiments, the first mixture is heated for about for about 4 hours. In some embodiments, the first solvent is removed from the first extract by evaporation. In some embodiments, the evaporation takes place at a temperature from about 40° C. to about 60° C. In some embodiments, the second extraction comprises the steps of: mixing the first extract with a second solvent to produce a second mixture; allowing the second mixture to partition into an aqueous layer and a non-aqueous layer; collecting the non-aqueous layer; removing any non-active compounds; and removing the second solvent to obtain a purified extract of phytochemicals. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation.

In some embodiments, the composition further comprises at least one additional ingredient selected from the group consisting of: active pharmaceutical ingredients; enhancers; excipients; and agents used to adjust the pH, buffer the composition, prevent degradation and improve appearance, odor or taste.

In some embodiments, the invention comprises a method of isolating an extract of Astragalus membranaceus, Astragalus mongholicus, Anemarrhenae asphodeloides and Achyranthes bidentata.

In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone.

In some embodiments, the purified extract of phytochemicals is obtained by performing a first extraction and a second extraction.

In some embodiments, the first extraction comprises the steps of: dispersing Astragalus membranaceus, Astragalus mongholicus, Anemarrhenae asphodeloides and Achyranthes bidentata in a first solvent to form a first mixture; heating the first mixture; and removing the first solvent to obtain a first extract. In some embodiments, the first solvent is a solution of ethanol and water. In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. In some embodiments, the first mixture is heated for about 2 hours to about 6 hours. In some embodiments, the first mixture is heated for about for about 4 hours. In some embodiments, the first solvent is removed from the first extract by evaporation. In some embodiments, the evaporation takes place at a temperature from about 40° C. to about 60° C. In some embodiments, the second extraction comprises the steps of: mixing the first extract with a second solvent to produce a second mixture; allowing the second mixture to partition into an aqueous layer and a non-aqueous layer; collecting the non-aqueous layer; removing any non-active compounds; and removing the second solvent to obtain a purified extract of phytochemicals. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation.

In some embodiments, the invention comprises a method of treating a subject with a disorder which may be treatable with a composition comprising a therapeutically effective amount of an extract of Astragalus membranaceus, Astragalus mongholicus, Anemarrhenae asphodeloides and Achyranthes bidentata.

In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone.

In some embodiments, the purified extract of phytochemicals is obtained by performing a first extraction and a second extraction.

In some embodiments, the first extraction comprises the steps of: dispersing Astragalus membranaceus, Astragalus mongholicus, Anemarrhenae asphodeloides and Achyranthes bidentata in a first solvent to form a first mixture; heating the first mixture; and removing the first solvent to obtain a first extract. In some embodiments, the first solvent is a solution of ethanol and water. In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. In some embodiments, the first mixture is heated for about 2 hours to about 6 hours. In some embodiments, the first mixture is heated for about for about 4 hours. In some embodiments, the first solvent is removed from the first extract by evaporation. In some embodiments, the evaporation takes place at a temperature from about 40° C. to about 60° C.

In some embodiments, the second extraction comprises the steps of: mixing the first extract with a second solvent to produce a second mixture; allowing the second mixture to partition into an aqueous layer and a non-aqueous layer; collecting the non-aqueous layer; removing any non-active compounds; and removing the second solvent to obtain a purified extract of phytochemicals. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation.

In some embodiments, the composition further comprises at least one additional ingredient selected from the group consisting of: active pharmaceutical ingredients; enhancers; excipients; and agents used to adjust the pH, buffer the composition, prevent degradation and improve appearance, odor or taste.

In some embodiments, the composition is administered as a dosage form selected from the group consisting of: a solid oral dosage form, liquid oral dosage form, gelatin capsule dosage form, a vaginal suppository, a rectal suppository or a spray.

In some embodiments, the therapeutically effective amount is an amount which elicits an estrogenic effect.

In some embodiments, the estrogenic effect is selected from the group consisting of: treating or preventing at least one climacteric symptom; treating or preventing osteoporosis; or any combination thereof. In some embodiments, the estrogenic effect is treating or preventing at least one climacteric symptom. In some embodiments, the climacteric symptom is selected from the group consisting of: hot flashes, insomnia, vaginal or vulvar atrophy, decreased libido, urinary incontinence, headache, depression or any combination thereof. In some embodiments, the climacteric symptom is vaginal or vulvar atrophy. In some embodiments, the estrogenic effect includes treating or preventing osteoporosis.

In some embodiments, the invention comprises a method of activating a gene under control of an estrogen response element, comprising administering to a cell having an estrogen response element operatively linked to the gene and an estrogen receptor an amount of a composition, wherein the composition comprises an extract of Astragalus membranaceus, Astragalus mongholicus, Anemarrhenae asphodeloides and Achyranthes bidentata and wherein the extract is in an amount sufficient to activate the gene.

In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone.

In some embodiments, said cell is in vitro. In some embodiments, said cell is in vivo.

In some embodiments, said cell is in an ERα+ breast tissue. In some embodiments, said cell is in an ERβ+ breast tissue. In some embodiments, said cell is in an ERα+/ERβ+ breast tissue.

In some embodiments, said estrogen response element is expressed in a transformed cell. In some embodiments, both the estrogen response element and the estrogen receptor are expressed in the cell. In some embodiments, said estrogen response element is heterologously expressed in the cell. In some embodiments, both the estrogen response element and the estrogen receptor are heterologously expressed in the cell.

In some embodiments, said cell is selected from the group consisting of a U937, a U2OS, a MDA-MB-435 and a MCF-7 cell transformed with an ERE-controlled gene. In some embodiments, the cell expresses ERα. In some embodiments, the cell expresses ERβ. In some embodiments, the ERE-controlled gene is ERE-tk-Luc.

In some embodiments, the invention comprises a method of repressing expression of a TNF RE-controlled gene, comprising administering to a cell comprising a gene under control of a TNF response element and an estrogen receptor an amount of a composition, wherein the composition comprises an extract of Astragalus membranaceus, Astragalus mongholicus, Anemarrhenae asphodeloides and Achyranthes bidentata and wherein the extract is in an amount sufficient to repress the TNF RE-controlled gene.

In some embodiments, the phytochemical is selected from the group consisting of: calycosin, nyasol and tetracyclic isoflavone. In some embodiments, the extract comprises at least two of: calycosin, nyasol and tetracyclic isoflavone. In some embodiments, the extract comprises calycosin, nyasol and tetracyclic isoflavone.

In some embodiments, the TNF RE-controlled gene is TNF-α. In some embodiments, the TNF RE-controlled gene is TNF RE-Luc.

In some embodiments, the cell is in vitro. In some embodiments, the cell is in vivo.

In some embodiments, the cell is in an ER+ breast tissue. In some embodiments, the cell is in an ERα+ breast tissue. In some embodiments, the cell is in an ERβ+ breast tissue.

In some embodiments, the TNF response element is endogenously expressed in the cell. In some embodiments, both the TNF response element and the estrogen receptor are endogenously expressed in the cell. In some embodiments, the TNF response element is heterologously expressed in the cell. In some embodiments, both the TNF response element and the estrogen receptor are heterologously expressed in the cell

In some embodiments, the cell contains an estrogen receptor gene, is transformed with an TNF response element-controlled gene and is selected from the group consisting of a U937, a U2OS, a MDA-MB-435 and a MCF-7 cell. In some embodiments, the estrogen receptor gene is a gene expressing ERα. In some embodiments, the estrogen receptor gene is a gene expressing ERβ.

### DEFINITIONS

As used herein, an extract is a composition of matter prepared by contacting a solvent with plant matter under conditions suitable for drawing one or more phytochemical compounds from the plant matter into an extraction medium to form a solution and optionally diluting, fractionating, further purifying and/or concentrating the solution to form the extract. In some embodiments of the invention, said plant matter is a mixture of Astragalus membranaceus, Astragalus mongholicus, Anemarrhenae asphodeloides and Achyranthes bidentata. Thus, the term “extract” includes the dry residue remaining when a solution resulting from extraction of plant matter (Astragalus membranaceus, Astragalus mongholicus, Anemarrhenae asphodeloides and Achyranthes bidentata) with an extraction medium is concentrated (e.g. by evaporation) until only dry residue remains; the term “extract” also encompasses a concentrated solution that results from extraction of plant matter with an extraction medium, followed by concentration of the solution (e.g. by evaporation); the term “extract” also encompasses a diluted solution comprising a solution produced by contacting an extraction medium with plant matter and one or more diluents. The term “extract” can further include the product of an extraction step followed by one or more purification and/or fractionating steps.

The term “in vivo” refers to contacting or treatment within a living organism, such as a living human or other mammal, such as a mouse or rat.

As used herein, the terms “treat”, “treating” and “treatment” refer to a method of alleviating vaginal atrophy or one or more of the symptoms thereof.

As used herein, prophylaxis refers to the delay of onset, reduction in severity and/or reduction in frequency of one or more symptoms of a disease state. Thus, prophylaxis is not intended to embrace absolute prevention of a disease or disease state; nor is prophylaxis intended to imply prevention of onset of a disease state, although prophylaxis may in some embodiments include administration of a composition comprising an extract as described herein with the purpose of: reducing the likelihood that the patient will suffer a particular symptom or set of symptoms, reducing the severity or frequency of a symptom or set of symptoms in a patient who has already been diagnosed as, or is judged to be at risk for, suffering from the symptom or set of symptoms.

Specifically, treatment of menopause includes ameliorating, reducing the severity of and/or reducing the frequency of one or more symptoms of menopause, perimenopause or post-menopause. Additionally, it may include prophylactic administration of the invention composition and extract with the purpose and/or effect of delaying onset of one or more menopausal symptoms, reducing the severity or frequency of menopausal symptoms to a negligible level or both.

As used herein, palliation includes reduction in the severity, number and/or frequency of occurrences of a disease, disorder, syndrome, condition or symptom.

As used herein, “E₂” refers to estradiol, a naturally occurring estrogen.

As used herein, “administer”, “administering” or “administration” refers to the delivery of an extract or extracts of this invention or of a pharmaceutical composition containing an extract or extracts of this invention to a patient in a manner suitable for the treatment of vaginal atrophy. The term includes self-administration and administration by a health care professional or other care provider.

A “patient” herein specifically refers to a menopausal or perimenopausal woman who either is suffering from vaginal atrophy or is judged by a competent medical practitioner to be likely to develop vaginal atrophy.

As used herein, the term “therapeutically effective amount” refers to an amount of extract that is suitable for treating vaginal atrophy.

As used herein, a “pharmaceutical composition” refers to a mixture of one or more of the extracts described herein with other chemical components, such as physiologically acceptable carriers and excipients. The purpose of a pharmaceutical composition is to facilitate administration of an extract or extracts of this invention to a patient.

As used herein, the term “pharmaceutically acceptable” means that the referenced agent or excipient is generally regarded as acceptable for use in a pharmaceutical composition.

As used herein, a “physiologically acceptable carrier” refers to a carrier or diluent that does not cause significant irritation to an organism and does not abrogate or unduly interfere with the biological activity and properties of the administered composition. In some embodiments, the physiologically acceptable carrier may enhance uptake of one or more components of an extract of the invention, without necessarily causing or bringing about a medicinal effect in the patient by itself.

As used herein, an “excipient” refers to an inert substance in a pharmaceutical composition, which facilitates administration of an extract of this invention. Thus, the term “excipient” specifically excludes other active ingredients, such as, in particular, other menopause- or vaginal atrophy-treating agents, such as hormones. Illustrative, non-limiting examples of inert excipients include flavorings, taste-masking agents, sweeteners, binders, disintegrants, glidants, diluents, and combinations of two or more thereof.

As used herein, the terms “comprising”, “comprises”, “comprise” and grammatical variants thereof are inclusive or open-ended and do not exclude additional, unrecited elements or method steps. The terms “include”, “includes”, “contain”, “contains”, “containing” and grammatical variants thereof are likewise inclusive.

As used herein, the phrase “consisting of” excludes any element, step, or ingredient not specified in the following portion of the sentence.

As used herein, the phrase “consisting essentially of” limits the scope of the following part of the sentence to the specified materials or steps and those that do not materially affect the basic and novel characteristic(s) of the claimed invention.

As used herein, the term “grams dry weight per day” (also “gm dry weight”) means, in reference to an extract in accordance with the invention, the dry weight, in grams, of the residue after a quantity of herb has been extracted and the extraction medium has been removed, e.g. by evaporation or freeze drying.

As used herein “tetracyclic isoflavone” (“TCIF”) means a compound having the following structure:

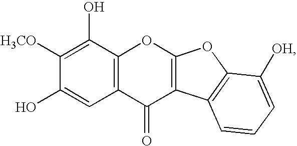

or a pharmaceutically acceptable salt thereof.

### Estrogen

Estrogens are a group of steroidal compounds. The most prevalent naturally occurring estrogens are estradiol, estriol and estrone. Through menopause, the estrogen is 17β-estradiol is most prevalent. In postmenopausal women estrone is most prevalent.

Follicles in the ovaries, the corpus luteum and the placenta are responsible for most of the estrogen produced in the female body. In addition, the liver, adrenal glands and the breasts produce estrogens, though in much smaller amounts. During and following menopause, these secondary sources become the dominant sites for the production of estrogen.

Estrogens can activate or repress the transcription of certain genes. There are two characterized pathways for activation of gene transcription, the classical ERE (estrogen response element) pathway and the AP-1 pathway.

### Estrogen Receptors

There are two known estrogen receptors, ERα and ERβ, which are members of the steroid nuclear receptor superfamily. ERα was first cloned in 1986; about 10 years later a second ER was discovered, which is referred to in the art as ERβ.

Structure of ERs

Both receptors are modular proteins made up multiple domains. The amino-terminus domain (A/B domain) is the least conserved region, exhibiting only a 15% homology between ERα and ERβ. This domain harbors an activation function (AF-1) that can activate gene transcription activation in the absence of estradiol.

The central region of ERs contains two zinc finger motifs that bind to an inverted palindromic repeat sequence separated by three nucleotides located in the promoter of target genes.

The DNA binding domain (DBD) in ERα and ERβ are virtually identical, exhibiting 95% homology.

The carboxy-terminus domain contains the ligand binding domain (LBD), which carries out several essential functions. The LBD contains a region that forms a large hydrophobic pocket where estrogenic compounds bind, as well as regions involved in ER dimerization. The LBD also contains a second activation function (AF-2) that interacts with coregulatory proteins. AF-2 is required for both estrogen activation and repression of gene transcription. The LBDs of ERα and ERβ are only about 55% homologous.

The striking differences in the amino acid composition of the ERα and ERβ LBDs may have evolved to create ERs that have distinct transcriptional roles. This would permit ERα and ERβ to regulate the activity of different genes and to elicit different physiological effects. This notion is supported by studies of ERα and ERβ knockout mice. For example, the ERα knockout mice have primitive mammary and uterine development, whereas the ERβ knockout mice develop normal mammary glands and uterus. These observations demonstrate that only ERα is required for the development of these tissues. Furthermore, ERα is more effective than ERβ at activating genes, whereas ERβ is more effective than ERα at repressing gene transcription.

Method of Action

Estrogen receptors are present in many tissues and are involved in variety of physiological regulatory effects as well as developmental effects. Different tissues have varying levels of expression of the two known estrogen receptors subtypes, ERα and ERβ. Both estrogen receptors are expressed in the vagina, bone, brain and breast tissues, while the uterus expresses mostly ERα.

Functionally, estrogen activates and represses gene transcription on both receptors, but only 24% of the genes they regulate are the same for ERα and ERβ. Estrogen significantly increases the proliferation of MCF-7 breast cancer cells containing only ERα. When the same MCF-7 breast cancer cells are infected with ERβ, it inhibits their proliferation and arrests their growth. Furthermore, estrogen treatment of the ERβ infected MCF-7 breast cancer cells results in further inhibition of proliferation

Estrogens can activate or repress gene transcription. There are two characterized pathways for activation of gene transcription, the classical ERE (estrogen response element) pathway and the AP-1 pathway. There are at least three essential components necessary for estrogens to regulate the transcription of genes: the ERs (ERα and/or ERβ), the promoter element in target genes and coregulatory proteins. The binding of estradiol to the ER leads to a conformational change, which results in several key steps that initiate transcriptional pathways. First, the interaction of E₂ with ER leads to the dissociation of chaperone proteins; this exposes the ER's dimerization surface and DNA binding domain. Loss of the chaperone proteins allows the ERs to dimerize and bind to an ERE in the promoter region of a target gene.

Second, the binding of E₂ moves helix 12 of the ER's LED to create a surface that assembles the AF-2 function of the ER. The AF-2 consists of a conserved hydrophobic pocket comprised of helices 3, 5 and 12 of the ER, which together form a binding surface for the p160 class of coactivator proteins (coactivators), such as steroid receptor coactivator-1 (SRC-1) or glucocorticoid receptor interacting protein 1 (GRIP 1). Coactivators (also known as “coregulators”) contain several repeat amino acid motifs comprised of LXXLL, which project into hydrophobic cleft surrounded by the AF-2's helices. The coactivators possess histone acetylase activity. It is thought that gene activation occurs after the ERs and coactivator proteins form a complex on the ERE that causes the acetylation of histone proteins bound to DNA. The acetylation of histones changes the chromatin structure so that the ELR/coregulator complex can form a bridge between the ERE and basal transcriptional proteins that are assembled at the TATA box region of the target gene to initiate gene transcription.

### Estrogen Replacement Therapy

Until very recently, postmenopausal estrogen replacement was widely used to treat the symptoms of vaginal atrophy. Systemic estrogen preparations such as oral estrogen pills and transdermal estrogen patches were favored by those seeking to alleviate other menopausal symptoms such as hot flashes or to prevent postmenopausal bone density loss. Over the past decade, however, randomized clinical trials have conclusively shown that systemic estrogen replacement increases the risk of cardiovascular and venous thromboembolic disease, in addition to endometrial hyperplasia and estrogen-responsive cancers. Furthermore, estrogen replacement appears to increase the risk of other urogenital problems such as urinary incontinence that are already widespread in older women with vaginal atrophy. In the Women's Health Initiative trials, for example, women taking unopposed estrogen had a 52% higher risk of developing urinary incontinence compared to placebo and women taking combined estrogen plus progestin had a 39% higher risk compared to placebo.

Vaginal estrogen therapy in the form of vaginal creams, vaginal rings and slow-release vaginal tablets has also been shown to be effective in treating vaginal dryness. Because these therapies result in lower serum estradiol levels than systemic estrogen preparations, they are thought to pose a lower risk of cardiovascular disease, venous thromboembolism, invasive breast cancer and endometrial hyperplasia. Nevertheless, systemic absorption of estrogen does occur with vaginal estrogen administration, especially at the beginning of treatment when the vaginal epithelium is thin and atrophic. As a result, women using vaginal estrogen (100 to 400 mcg of estradiol per day) must simultaneously take a progestin to prevent endometrial hyperplasia.

While lower-dose vaginal estrogen therapies (10 to 100 mcg of estradiol per day) are thought to produce only minimal changes in serum estradiol levels, some investigators believe that a “first pass” effect can occur in which vaginal estrogen travels to the uterus via local veins and exerts a greater effect there than would occur from systemic delivery. As a result, many clinicians still prefer to avoid long-term use of these therapies in women with a higher-than-average risk of developing endometrial cancer. Several small studies have suggested that endometrial proliferation may occur in women treated with low-dose vaginal estrogen, although other trials have documented no proliferation with these therapies.

### Selective Estrogen Replacement Modulators (SERMS)

The known SERMs antagonize and agonize the action of the estrogen receptors. Their unique pharmacological action allows the distinct SERMs to exert their action through tissue selectivity, where they either antagonize, as in the case of breast tissue or agonize as in the case of bone tissue. Their action is through both estrogen receptors and none of the currently available SERMs exhibit selectivity to one of the two known estrogen receptors.

Selective estrogen receptor modulators (SERMs), which have estrogenic effects on bone but not on the breast or uterus, have also been explored as potential treatments for the symptoms and complications of vaginal or vulvar atrophy. One double-blind, placebo-controlled trial of the SERM raloxifene found that a smaller proportion of women treated with the drug versus placebo required surgery for urogenital prolapse. Several small randomized trials found improvements in vaginal maturation indices and vaginal pH in women taking the SERM lasofoxifene relative to placebo. Because some SERMs cause down regulation of estrogen receptors in the urogenital tract, they also have the potential to worsen symptoms of vaginal or vulvar atrophy. Furthermore, SERMs can worsen the severity of other bothersome menopausal symptoms such as hot flashes, which make them less attractive to menopausal women for treatment of vaginal atrophy.

Effect of SERMs on the ERE Pathway

Unlike estrogens, SERMs do not activate the ERE pathway. Instead, the SERMs competitively block the effects of estrogens on the ERE pathway. Like estrogens, SERMs bind to ERα and ERβ with high affinity and cause the dissociation of chaperone proteins, ER dimerization and binding of ERs to the ERE. Thus, the antagonist action of SERMs occurs at a step distal to the binding of the ER to the promoter region. The molecular mechanism of the antagonist action of the SERMs has been clarified by the crystallization of the ERα and ERβ LBDs. It is clear from the structure of the ER LBDs that E₂, tamoxifen and raloxifene bind to the same binding pocket. However, tamoxifen and raloxifene contain a bulky side-chain that is absent in E₂. The ER x-ray structures have revealed that the bulky side chain of SERMs obstructs the movement of the LBD, which prevents the formation of a functional AF-2 surface. Remarkably, when a SERM binds to ERα a sequence (LXXML) in helix 12, which is similar to the LXXLL motif, interacts with the hydrophobic cleft of the AF-2 surface to occlude the coactivator recognition site. Thus, unlike estrogens, SERMs do not create a functional AF-2 surface; this prevents the binding of coactivators. Because the coactivator proteins do not bind to the AF-2 surface in the presence of SERMs, the activation pathway is abruptly halted. Instead of recruiting coactivator, ERs liganded with SERMs recruit corepressors, such as N-CoR.

These studies demonstrated that the antagonist properties of SERMs are due to at least three factors. First, SERMs bind to the same binding pocket as estrogens and competitively block their binding to the ERs. Second, SERMs prevent ER from interacting with coactivator proteins that are required for transcriptional activation of the ERE pathway. Third, SERMs recruit corepressors, which prevent transcriptional activation of genes. These actions of SERMs most likely explain how raloxifene and tamoxifen act as antagonists in breast cells to inhibit development of breast cancer.

SERMs are also more effective than E₂ at activating genes with an AP-1 element. In fact, E₂ is an antagonist of SERM-mediated activation of AP-1 elements. It has been postulated that SERMs exhibit agonistic actions in tissues, such as the bone and endometrium by activating the AP-1 pathway. Interestingly, SERMs are more potent at activating the AP-1 pathway in the presence of ERβ, which indicates that SERMs will trigger the AP-1 pathway more efficiently in tissues that are rich in ERβ. The role of the AP-1 pathway in estrogen-mediated breast carcinogenesis is unclear, because estrogens are much weaker at activating the AP-1 pathway compared to SERMs. However, it has been proposed that the AP-1 pathway may be involved in resistance to tamoxifen in breast tumors.

In accordance with aspects of the present invention, studies have been performed, which demonstrate that: ERβ is weaker than ERα at activating ERE-tkLuc; ERβ is more effective than ERα at repressing the TNF-RE-tkLuc; and that ERβ inhibits ERα-mediated transcriptional activation of ERE-tkLuc. Detailed experiments are discussed in the Examples section hereinafter.

### Vaginal and Vulvar Atrophy

The onset of menopausal vaginal atrophy is associated with the decline of circulating estrogens. From perimenopause to postmenopause, serum estrogen levels have been shown to drop from approximately 400 pg/ml to less than 10 pg/ml. Estrogen replacement treatment has been used successfully for the treatment of vaginal dryness. It is hypothesized that the effect is accomplish via agonistic stimulation of the estrogen receptors in the vulva and vaginal canal.

Definitions and Epidemiology

Vaginal symptoms, including dryness, irritation, itching, bleeding and dyspareunia increase as women transition through the menopause, with about 30% of peri- and early postmenopausal women and up to 47% of later postmenopausal women expressing these complaints. The natural history of vaginal complaints is not entirely clear, but unlike other menopausal symptoms such as hot flushes, which generally improve over time; these symptoms generally persist and may even worsen with aging.

Postmenopausal vaginal symptoms are generally associated with physical examination findings that include pallor, dryness, friability and decreased rugosity of the vaginal mucosa. Compared to premenopausal women, postmenopausal women with vaginal symptoms generally have decreased vaginal blood flow and secretions, hyalinization of collagen, fragmentation of elastin and proliferation of vaginal connective tissue. Vaginal epithelial cells demonstrate decreased maturation and ability to store glycogen and secrete hydrogen ions to maintain lower pH levels. Quantitative vaginal epithelial cell cytology generally reveals that the proportion of vaginal epithelial cells that are parabasal (immature) is greater than 20%, compared to less than 5% in premenopausal women. Vaginal fluid, which is acidic in premenopausal women, becomes more neutral, typically with a pH of less than 5.0.

The constellation of symptoms, physical findings and other changes observed in older women is generally referred to as vaginal atrophy or atrophic vaginitis. Urinary symptoms such as urgency and dysuria may also occur when vaginal atrophy is accompanied by atrophy or inflammation of the adjacent tissues of lower urinary tract (vaginal or vulvar atrophy). Treatment with estrogen improves many of the cytologic and physiologic changes in the vagina, suggesting that estrogen deficiency plays a major role in the etiology of these changes. However, while lower serum androgen levels correlate with vaginal symptoms, estrogen levels do not and some of the cytologic and physiologic changes of the postmenopausal vagina can be improved with use of a non-estrogen containing vaginal moisturizer.

Unlike other menopausal symptoms such as hot flashes and night sweats, vaginal symptoms frequently develop more than 5 years after the onset of menopause and persist for decades after women cease to have regular menstrual cycles. Epidemiologic studies suggest that vaginal dryness affects 3% to 22% of premenopausal women, 7% to 39% of perimenopausal women and 17% to 47% of postmenopausal women. While the majority of studies on vaginal atrophy have been conducted in white women, the prevalence of vaginal dryness may be higher in Black and Latina women.

Pathophysiology

During the reproductive years, it has been demonstrated that estrogen plays an important role in maintaining the acidity of the vaginal fluids by promoting proton secretion by the vaginal epithelial cells. It has also been shown that estrogen stimulates the vaginal epithelial cells to produce and store glycogen, which is in turn metabolized into lactic acid by the lactobacilli that normally colonize the vagina wall. The resulting low vaginal pH (<5.0) serves to inhibit the growth of pathogenic Gram-negative bacteria that serve as a reservoir for vaginal and urinary tract infections. Estrogen is also thought to play a role in maintaining the composition and cross-linking of collagen in the connective tissues of the vaginal wall, although the exact mechanism for this is not well understood.

During menopause and other conditions associated with a significant decrease in circulating estrogen levels (e.g. oophorectomy, anti-estrogenic medications, immunologic disorders), the normal function of the vaginal epithelial cells is disrupted. In the setting of decreased glycogen production and proton secretion, the vaginal pH increases to 6.0-7.0. Other changes in the vaginal mucosa associated with either estrogen deprivation or aging include proliferation of connective tissue, fragmentation of elastin and hyalinization of collagen, resulting in increased friability of the vaginal wall.

Astragalus membranaceus and Astragalus mongholicus

The plants Astragalus membranaceus and Astragalus mongholicus are collectively referred to as Radix astragali. Radix Astragali is the dried root of Astragalus membranaceus (Astragalus mongholicus. The herb is also referred to by the Chinese name (Huang Qi) and the common names astragalus root, hoang ky, huang-chi, huanggoi, huangqi, hwanggi, membranous milk vetch, milkvetch, Mongolian milk-vetch, neimeng huangqi, ogi, ougi and zhongfengmaomaitong.

Radix Astragali is a perennial plant, about 16 -36 inches tall. Radix Astragali is indigenous to China, the Democratic Peoples Republic of Korea, Mongolia and Siberia. The plant is commercially cultivated in northern China and the Democratic Peoples Republic of Korea and is harvested in the spring and autumn. The root is removed from fibrous root and rootstock and then dried in the sun.

The root is cylindrical with some branches relatively thick, 30-90 cm long 1-3 cm in diameter. The root surface color is pale brownish yellow or pale brown to dark brown with irregular, longitudinal wrinkles or furrows. The texture is hard and tenacious, broken with difficulty, fracture highly fibrous and starchy. The bark is yellowish white. The wood is pale yellow with radiate striations and fissures. The center of the wood is cork like, occasionally looking like rotten wood, blackish brown or hollowed.

Anemarrhenae asphodeloides

Rhizomae Anemarrhenae asphoeloides Bunge of the Liliaceae (a.k.a. Asphodelaceae) family is also referred to by the Chinese name Zhi Mu. No additional common names have been identified. Anemarrhenae asphoeloides is an evergreen perennial growing to 0.5 m by 1 m. It is in flower from August to September. The flowers are hermaphrodite (have both male and female organs).

The plant is commercially cultivated in the northern Chinese provinces of Hebei, Shanxi and Inner Mongolia. The plant is commercially cultivated and harvested in spring and autumn. The rhizome is removed from the rootlets and then dried in the sun.

The root is long, flat, angled with few branches. The root surface color is light to dark brown the center of the root has longitudinal grooves that end in a tight node. The root has yellowish fibrous leaf bases, with one being slightly raised, with wrinkles and spotted root and fibrous scars. The texture is hard and brittle, easily broken, fracture highly fibrous and starchy. The wood is ivory colored. The center of the wood is cork-like.

Achyranthes bidentata

Radix Achyranthes bidentata B1. of the Amarathaceae family is also known by the Chinese name Huai Niu Xi and the common names two-toothed amaranthus and oxknee. Achyranthes bidentata is an erect perennial with slender rambling branches, elliptical leaves and greenish white flowers on terminal spikes grows up to 1 m tall.

Achyranthes bidentata is commercially cultivated mainly in Henan province. The plant is harvested in the summer. The aerial part is cut from the root and only the root is used. The root is dried in the sun.

The root is cylindrical, 15-50 cm long 0.4-1 cm in diameter. The surface color is grey yellow with longitudinal wrinkles and tiny-long lenticles. The texture is hard and brittle, easily broken. The bark is yellowish white. The cross section is smooth and yellow-brown in color with pith in the center. The vascular bundle is visible in the cross section.

### Calycosin

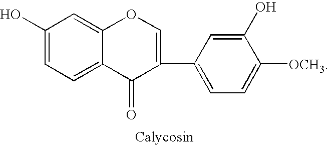

Calycosin is an isoflavonoid with selective estrogen receptor beta activity. It is extracted from Astragalus membranaceus and Astragalus mongholicus. The chemical formula for calycosin is C₁₆H₁₂O₅. The molecular weight of calycosin was measured as being 284. Studies have shown that it also causes ERβ selective transcriptional activation and does not have an antagonistic effect on either receptor. Calycosin may be present in the pharmaceutical composition in the form of a pharmaceutically acceptable salt, although calycosin is considered of sufficient solubility in its free form (non-salt) that in certain preferred embodiments, it is not deemed necessary for calycosin to be present in the composition in a salt form. Nonetheless, where “calycosin” appears herein, the term is intended to include both free and salt forms, unless otherwise specified.

### Nyasol

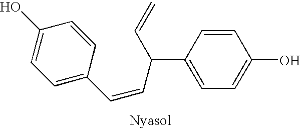

Nyasol is a phenylpropanoid with selective estrogen receptor beta activity. It is isolated from Anemarrhenae asphodeloides. The chemical formula for nyasol is C₁₇H₁₆O₂. The molecular weight of nyasol was measured as being 252. Studies have shown that while nyasol binds with equal affinity to both estrogen receptor subtypes, it results in ERβ transcriptional activation only. Additionally, it has been shown that it does not have an antagonistic effect on either receptor. Nyasol may be present in the pharmaceutical composition in the form of a pharmaceutically acceptable salt, although nyasol is considered of sufficient solubility in its free form (non-salt) that in certain preferred embodiments, it is not deemed necessary for nyasol to be present in the composition in a salt form. Nonetheless, where “nyasol” appears herein, the term is intended to include both free and salt forms, unless otherwise specified.

Tatracyclic isoflavone (TCIF)

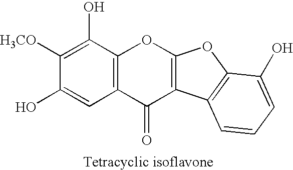

TCIF is an isoflavonoid with selective estrogen receptor beta activity. It is extracted from Achyranthes bidentata. The chemical formula for TCIF is C₁₆H₁₀O₇. The molecular weight of TCIF was measured as being 314. Studies have shown that it also causes estrogen receptor beta (ERβ) selective transcriptional activation. Additionally, it has been shown that it does not have an antagonistic effect on either receptor. TCIF (tetracyclic isoflavone) may be present in the pharmaceutical composition in the form of a pharmaceutically acceptable salt, although tetracyclic isoflavone is considered of sufficient solubility in its free form (non-salt) that in certain preferred embodiments, it is not deemed necessary for tetracyclic isoflavone to be present in the composition in a salt form. Nonetheless, where “tetracyclic isoflavone” (or “TCIF”) appears herein, the term is intended to include both free and salt forms, unless otherwise specified.

### Method of Extraction

In some embodiments, the invention comprises a method of isolating a purified extract of phytochemicals from the plant species Astragalus membranaceus, Astragalus mongholicus, Anemarrhenae asphodeloides and Achyranthes bidentata. In some embodiments, the purified extract of phytochemicals is obtained by performing a first extraction and a second extraction.

In some embodiments, the first extraction comprises the steps of: dispersing Astragalus membranaceus, Astragalus mongholicus, Anemarrhenae asphodeloides and Achyranthes bidentata in a first solvent to form a first mixture; heating the first mixture; and removing the first solvent to obtain a first extract. In some embodiments, the first solvent is a solution of ethanol and water.

When the plants are dispersed in the first solvent, the invention contemplates the use of the whole plant or any part or parts of the plant, such as the root, bark, wood, leaves, flowers (or flower such as: sepals, petals, stamens, pistils, etc.), fruit, seeds and/or parts or mixtures of any of the foregoing. Furthermore, the plants or parts thereof may be fresh cut, dried (including freeze dried), frozen, etc. Additionally, the invention optionally comprises first comminuting the plant matter in order to increase its surface area to volume ratio and to concomitantly increase efficiency of the extraction process. Methods of comminuting plant matter include grinding, chopping, blending, shredding, pulverizing, triturating, etc.

In some embodiments, the first mixture is heated to a temperature from about 10° C. to about 60° C. In some embodiments, the first mixture is heated to a temperature from about 15° C. to about 50° C. In some embodiments, the first mixture is heated to a temperature from about 20° C. to about 40° C. The person of skill in the art will recognize that the proper balance should be drawn between extraction efficiency on the one hand and phytochemical stability on the other.

Once the solvent and the plant matter are combined, they are optionally agitated to ensure efficient exchange of estrogenic compound from the plant matter into the extraction medium and are left in contact for a time sufficient to extract a useful amount of phytochemical compound from the plant matter into the extraction medium. In some embodiments, the first mixture is heated for about 2 hours to about 6 hours. In some embodiments, the first mixture is heated for about for about 4 hours. After this time, the first extract is separated from the plant matter. Such separation is accomplished by an art-recognized method, e.g. by filtration, decanting, etc.

In some embodiments, the first solvent is removed from the first extract by evaporation. In some embodiments, the evaporation takes place at a temperature from about 40° C. to about 60° C.

In some embodiments, the second extraction comprises the steps of: mixing the first extract with a second solvent to produce a second mixture; allowing the second mixture to partition into an aqueous layer and a non-aqueous layer; collecting the non-aqueous layer; removing any non-active compounds; and removing the second solvent to obtain a purified extract of phytochemicals. In some embodiments, the second solvent is a solution of ethanol and ethyl acetate. In some embodiments, the non-aqueous layer is dried after it is collected. In some embodiments, any non-active compounds are removed by filtering the dried non-aqueous layer over silica. In some embodiments, the silica is suspended in ethyl acetate. In some embodiments, the second solvent is removed from the purified extract of phytochemicals by evaporation.

Where water is the extraction solvent, purified water is suitable. Purified water includes distilled water, deionized water, water for injection, ultrafiltered water and other forms purified of water.

The ethanol that is employed in some embodiments of the invention is grain ethanol. Particularly, it is undenatured ethanol (e.g. pure ethanol, optionally containing some water, e.g. up to about 10% water).

The ethyl acetate that is utilized in some embodiments of the invention is an ester derived from ethanol and acetic acid. It is preferably pure ethyl acetate, though it may contain some water.

In some embodiments, a pure extract may be combined with one or more organic solvents. Such organic solvents may be of various polarities. In some embodiments, suitable solvents include ethyl ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether and other solvents, including those that are pharmaceutically acceptable and those that are generally regarded as safe (GRAS) for human consumption.

Where the extract is intended for pharmaceutical use, any additional ingredients are pharmaceutically acceptable. Where the compositions according to the invention are intended for use in assays or other uses that are not directed toward a living body, the additional ingredient(s) may be either pharmaceutically acceptable or not.

### Formulation of the Composition

A composition according to the invention includes an inventive plant extract or a composition comprising an inventive plant extract of the invention. In such embodiments, the inventive composition will optionally contain one or more additional ingredients. Such additional ingredients may be inert or active. Inert ingredients include solvents, excipients and other carriers. Active ingredients include active pharmaceutical ingredients (APIs), including those that exhibit synergistic activity in combination with the inventive plant extract.

In some embodiments, the compositions comprise pure extracts or combinations of extracts with one or more additional solvents. In some embodiments, the extract includes a partitioned or further purified extract. Partitioning or purification may be conducted using various separation techniques, including chromatography. In some embodiments, the extract is a purified or partitioned extract obtained by means of anion exchange chromatography, cation exchange chromatography, reverse phase chromatography, normal phase chromatography, affinity chromatography or exclusion chromatography, to further concentrate active agents in the extract. In some embodiments, the purified or partitioned extract is obtained via one or more steps of liquid chromatography, such as high performance liquid chromatography (HPLC). In some embodiments, high performance liquid chromatography is preparative scale high performance liquid chromatography. In some embodiments, the HPLC is reverse phase or ion exchange chromatography. Other means of separation may also be used to purify or partition the extract, including separation in a separatory funnel or other bi- or multi-phasic separatory mechanism.

In some embodiments, the purified or partitioned extract may be combined with one or more additional active or inactive ingredients, such as solvents, diluents, etc. In some embodiments, suitable solvents may include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether and other solvents, including those that are pharmaceutically acceptable and those that are generally regarded as safe (GRAS) for human consumption.

Suitable additional ingredients include solvents. Solvents may be subdivided into pharmaceutically acceptable and non-pharmaceutically acceptable solvents. In this context, it is to be understood that some pharmaceutically acceptable solvents include water for injection (WFI), which may be pH adjusted and/or buffered to a preselected pH or pH range, e.g. from about 2 to about 8, more specifically from about 4.0 to about 7.5 and more particularly from about 4.9 to about 7.2.

Pharmaceutically acceptable solvents may further comprise one or more pharmaceutically acceptable acids, bases, salts or other compounds, such as carriers, excipients, etc. Pharmaceutically acceptable acids include HCl, H₂SO₄ H₃PO₄, benzoic acid, etc. Pharmaceutically acceptable bases include NaOH, KOH, NaHCO₃, etc. Pharmaceutically acceptable salts include NaCl, NaBr, KCl, etc. Acids and bases may be added in appropriate proportions to buffer a pharmaceutically acceptable solution at a particular, pre-selected pH, especially a pH in the range of about 2-8, more especially in the range of about 5.0 to about 7.2

In some embodiments, the composition further comprises at least one additional ingredient selected from the group consisting of: active pharmaceutical ingredients; enhancers; excipients; and agents used to adjust the pH, buffer the composition, prevent degradation and improve appearance, odor or taste. Examples of coloring agents include β-carotene, Red No. 2 and Blue No. 1. Examples of preservatives include stearic acid, ascorbyl stearate and ascorbic acid. Examples of corrigents include menthol and citrus perfume.

In some embodiments, the drug delivery system of the invention may advantageously comprise an absorption enhancer. The term “enhancer”, means any material which acts to increase absorption across the mucosa and/or increases bioavailability. In some embodiments, such materials include mucolytic agents, degradative enzyme inhibitors and compounds which increase permeability of the mucosal cell membranes. Whether a given compound is an “enhancer” can be determined by comparing two formulations comprising a non-associated, small polar molecule as the drug, with or without the enhancer, in an in vivo or good model test and determining whether the uptake of the drug is enhanced to a clinically significant degree. The enhancer should not produce any problems in terms of chronic toxicity because in vivo the enhancer should be non-irritant and/or rapidly metabolized to a normal cell constituent that does not have any significant irritant effect.

In some embodiments, preferred enhancing materials lysophospholipids, for example lysophosphatidylcholine obtainable from egg or soy lecithin. Other lysophosphatidylcholines that have different acyl groups as well as lyso compounds produced from phosphatidylethanolamines and phosphatidic acid which have similar membrane modifying properties may be used. Acyl carnitines (e.g. palmitoyl-dl-carnitine-chloride) is an alternative. In some embodiments, a suitable concentration is from 0.02 to 20% w/v.

In some embodiments, enhancing agents that are appropriate include chelating agents (EGTA, EDTA, alginates), surface active agents (especially non-ionic materials), acyl glycerols, fatty acids and salts, tyloxapol and biological detergents listed in the SIGMA Catalog, 1988, page 316-321 (which is incorporated herein by reference). Also agents that modify the membrane fluidity and permeability are appropriate such as enamines (e.g. phenylalanine enamine of ethylacetoacetate), malonates (e.g. diethyleneoxymethylene malonate), salicylates, bile salts and analogues and fusidates. Suitable concentrations are up to 20% w/v.

### Use of the Composition in an Assay

Extracts according to the present invention provide estrogenic activation of genes under control of the estrogen response element (ERE). Accordingly, in some cells an inventive extract possesses estrogenic properties—i.e. contacting a cell comprising an ERE and an ER (ERα, ERβ or both) with an inventive extract gives rise to stimulation of a gene under control of the ERE. In an in vitro cell system, ERE-mediated activation by an inventive extract leads to expression of a gene that is operatively linked to the ERE. In particular embodiments, estrogenic interaction of an ER with an ERE linked to the minimal thymidine kinase promoter and the luciferase gene gives rise to enhanced luciferase expression. Thus, the extracts of the present invention may be used to identify ERα+ cell lines, ERβ+ cell lines and/or ERα+/ERβ+ cell lines having an ERE-containing promoter operatively linked to a reporter gene, such as luciferase. Extracts of the present invention may also be used as assay reagents, including standards, for identifying compounds having estrogenic effects in ER+ cell lines.

In one such assay method, an inventive extract is first prepared at a known activity or concentration. Quantification of the inventive extract is conveniently carried out by taring a container, measuring into the container a known volume of the extract, reducing the extract by evaporation or lyophilization to produce a residue and obtaining the mass of the container plus extract. The difference in mass between the container plus extract and the tare mass is the dry mass of the extract. The ratio of dry mass of extract per volume of extract is the concentration per unit volume. The extract may be used in its initial form, using the results of the foregoing quantitation method to specify its concentration. The residue can also be reconstituted by addition of water or another suitable solvent system to form an extract solution of known concentration.

Once the concentration of an extract is known, a standard curve is prepared. In general the ER+ cells are contacted with an extract and a signal relating to estrogenic activity is recorded. In particular, an ER+ cell has a reporter gene under the control of an ERE. This ER+ cell is contacted with an extract of the invention, which gives rise to a reporter signal in proportion to the amount of extract added. This step may be carried out with multiple samples at the same extract concentration, at different extract concentrations or both.

As an example, nine samples may be tested: the first three at a first concentration, the next three at a concentration that is a half log greater than the first and the next three at a concentration a whole log greater than first. The reporter signals are then observed and recorded and the resulting data points (plant extract concentration versus reporter signal strength) are fitted to a standard curve by a conventional curve-fitting method (e.g. least squares).

To evaluate the estrogenic effect of an extract, an extract is contacted with E+ cells having the reporter gene under control of the ERE. The reporter gene signal is observed and compared to the standard curve to quantitate an extract's relative estrogenic effect.

The ER+ cell line used in the foregoing method may be a cell line that naturally expresses ER, e.g. a human-derived ER+ breast cell carcinoma cell line. In some embodiments, the ER+ tissue is an immortalized human cell line, e.g. an immortalized bone marrow or breast cell line. Exemplary cell lines include human monocyte, osteoblast, malignant breast carcinoma and immortalized epithelial breast cell lines. Particular cell lines that may be mentioned include U937, U2OS, MDA-MB-435 and MCF-7 cell lines. Other ER+ cell lines, including immortalized cell lines, may also be used. Alternatively, the ER+ cell line may be a cell line that does not naturally express ER, such as a bacterial cell line, that has been transformed with an ER expression vector.

The ER+ cell line is transformed with a vector having a promoter containing an ERE that controls a reporter gene. For example, the vector may be a viral vector containing ERE, a minimal thymidine kinase promoter (tk) and a luciferase gene (Luc). The construct is transfected into the target cell by known methods and expression of the ER-ERE-tk-Luk system is confirmed by e.g. performing the foregoing assay on putative ER+ cells in the presence of known quantities of estradiol (E₂). Other methods of verifying successful transformation of ER+ cells include immunostaining with known ER antibodies.

The ERE-containing promoter is a DNA containing an ERE sequence and a promoter sequence. The promoter sequence is an art-recognized promoter sequence, such as the minimal thymidine kinase (tk) promoter sequence. Other ERE-containing promoters are possible and are within the scope of the instant invention. The ERE and promoter sequence operate together to control expression of the reporter gene. As described herein, the estrogenic compound (an inventive extract or E₂, for example) binds to the ER, giving rise to ER dimer and forming the AF-2 surface. The ER dimer then binds to the ERE, activating the gene under control of the promoter. In some embodiments, the ERE is directly upstream of (5′-to) the promoter, to which it is directly ligated.

The reporter gene is a gene which, when expressed, gives rise to a detectable signal. The luciferase gene is a suitable reporter gene because it gives rise to the protein luciferase, which generates a detectable light signal in the presence of a single reagent, luciferin. In particular, the cDNA of the luciferase gene is expressed to produce the 62 kDa enzymatic protein, luciferase. The luciferase enzyme catalyzes the reaction of luciferin and ATP in the presence of Mg²⁺ and oxygen to form oxyluciferin, AMP, pyrophosphate (PPi) and emitted light. The emitted light is yellow-green (560 nm) and may easily be detected using a standard photometer. Because ATP, O₂ and Mg²⁺ are already present in cells, this reporter gene only requires addition of the reagent luciferin to produce a detectable signal and is especially well-suited for use in assays of the present invention. Other reporter genes that may be mentioned as being available in the art include chloramphenicol transacetylase (CAT), neomycin phosphotransferase (neo) and beta-glucuronidase (GUS).

In some assay methods of the invention, it is useful to further characterize an extract by comparison with one or more estrogenic compounds, SERMs, etc. Such assay methods are performed essentially as described above, making the proper substitutions of standard estrogenic compound and/or SERMs for an inventive extract in the appropriate parts of the method.

Extracts according to the present invention also repress gene expression by the TNF RE-mediated pathway. In some cases, extracts of the invention repress gene expression in vitro, especially in cells having a reporter gene (e.g. the luciferase gene, Luc) under control of a TNF RE. In some cases, extracts of the invention repress expression of TNF-α, which is a cytokine produced primarily by monocytes and macrophages. This cytokine is found in synovial cells and macrophages in various tissues and has been strongly implicated in rheumatoid arthritis (RA). TNF-α is also expressed in other inflammatory diseases and also as a response to endotoxins from bacteria. As repressors of TNF expression via the TNF RE repressor pathway, extracts of the invention are of interest in the treatment of inflammatory disorders associated with elevated levels of TNF.

In some embodiments of the invention, a cell line is prepared, which expresses one or both of ERα and ERβ as well as a reporter gene under control of TNF RE. The TNF RE is generally upstream of (5′-to) the reporter gene and signal detection is carried out as previously described herein.

The foregoing cell TNF RE-containing cell system further contains one or more copies of an ER gene—i.e. ERα, ERβ or both. The ER+ cell line used in the foregoing method may be a cell line that naturally expresses ER, e.g. a human-derived ER+ breast cell carcinoma cell line. In some embodiments, the ER+ tissue is an immortalized human cell line, e.g. an immortalized bone marrow or breast cell line. Exemplary cell lines include human monocyte, osteoblast, malignant breast carcinoma and immortalized epithelial breast cell lines. Particular cell lines that may be mentioned include U937, U2OS, MDA-MB-435 and MCF-7 cell lines. Other ER+ cell lines, including immortalized cell lines, may also be used. Alternatively, the ER+ cell line may be a cell line that does not naturally express ER, such as a bacterial cell line that has been transformed with an ER expression vector.

In the presence of a predetermined amount of luciferin and in the absence of an estrogenic compound, e.g. E₂ or an extract of the invention, the cell system emits a yellow light (560 nm) at an intensity, called the “control intensity” or the “baseline intensity”. Light emission at 560 nm is conveniently quantified in optical density units (O.D._{560 nm}). Upon addition of an estrogenic compound, e.g. E₂ or one of the inventive extracts, the intensity of 560 nm light emissions is attenuated as compared to the control. Remarkably, in the presence of a SERM, such as tamoxifen or raloxifene, luciferase expression increases and 560 nm light emission intensity also increases. Thus, extracts of the invention are capable of inducing an estrogenic TNF RE-controlled repression of gene expression.

The TNF RE-containing cell system can be used in an assay method according to the invention. In the inventive assay methods, the attenuation of luciferase activity (i.e. decreased emission of 560 nm light), correlates with increased estrogenic activity, whereas activation of luciferase activity (i.e. increased emission at 560 nm), correlates with anti-estrogenic activity. Standard curves may be prepared using known quantities of the inventive extracts, as described herein. Such standard curves may be further augmented by using other known estrogenic or anti-estrogenic standards, such as E₂ or some other known estrogenic compound and/or an anti-estrogenic SERM such as tamoxifen or raloxifene.

Cells from the transformed E+ cell line are then exposed to a candidate compound, the luciferase signal observed and the signal compared to the previously prepared standard curve(s), as described herein. A compound that causes an increase of luciferase activity as compared to control (baseline), will be characterized as an anti-estrogenic SERM, whereas a compound that causes a decrease in luciferase activity versus control will be classified as estrogenic. The estrogenic or anti-estrogenic effect can then be quantified by comparing the degree of luciferase expression decrease or increase against the decrease brought about by an inventive extract and optionally the respective signal decrease or increase brought about by E₂, tamoxifen and/or raloxifene.

The invention provides in vivo estrogenic methods of using the inventive compositions. In general, in vivo methods comprise administering to a subject an amount of an inventive extract sufficient to bring about an estrogenic effect in the subject. The in vivo methods will give rise to estrogenic ERE-controlled gene activation, TNF RE-controlled gene repression (e.g. TNF-α repression) or both. Thus, the in vivo methods will give rise to varied positive phenotypic effects in vivo.

The subject may be a mammal, such as a mouse, rat, rabbit, monkey, chimpanzee, dog, cat or a sheep and is generally female. The subject may also be human, especially a human female. In some embodiments, the subject is a post-menopausal or post-oophorectomic female and is in need of estrogenic therapy. In such case, the subject may be suffering from climacteric symptoms, such as hot flashes, insomnia, vaginal dryness, decreased libido, urinary incontinence and depression. In other such cases, the subject may be susceptible to or suffering from, osteoporosis. Suitable in vivo methods include treatment and/or prevention of medical indications that are responsive to estrogen replacement therapy.

Treatment with the Compositions

Administration of the pharmaceutical compositions according to the present invention comprising Astragalus membranaceus, Astragalus mongholicus, Anemarrhenae asphodeloides and Achyranthes bidentata) will be via a commonly used administrative route so long as one or more of the extracts is available to target tissue via that route. Some administrative routes that may be mentioned include: oral, nasal, buccal, rectal, vaginal and/or topical (dermal). Alternatively, administration may be by orthotopic, intradermal, subcutaneous, intramuscular, intraperitoneal or intravenous injection. Such compositions would normally be administered as pharmaceutically acceptable compositions, described supra.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, or otherwise improved, by administering an extract of the invention to the subject. Treatment further includes administering to a patient an amount of an extract of Astragalus membranaceus, Astragalus mongholicus, Anemarrhenae asphodeloides and Achyranthes bidentata suitable for ameliorating, palliating, or otherwise improving improving, said disease, disorder, syndrome, condition or symptom. Treatment also includes the concomitant amelioration, palliation, or improvement of the disease, disorder, syndrome, condition or symptom. In some embodiments, treatment implies prevention or delay of onset of a disease, disorder, syndrome, condition or symptom (i.e. prophylaxis), prevention or delay of progression of a disease, disorder, syndrome, condition or symptom and/or reduction in severity of a disease, disorder, syndrome, condition or symptom. In the case of climacteric symptoms, treatment includes and palliation of various symptoms, including vaginal and/or vulvar atrophy.

Treatment of osteoporosis includes identifying a person, such as a post-menopausal woman, at risk for bone loss and administering a plant extract of the present invention to the woman, whereby bone loss is reduced in severity, delayed in onset or prevented. In some embodiments, treatment of osteoporosis can also include addition of bone mass.

Prevention (and its grammatical variants) of a disease, disorder, syndrome, condition or symptom includes identifying a subject at risk to develop the disease, disorder, syndrome, condition or symptom and administering to that subject an amount of an inventive extract sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy and administering an extract of the present invention to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis and administering an extract of the present invention to the woman, whereby the onset of bone loss is blocked or delayed.

Palliation includes reduction in the severity, number and/or frequency of occurrences of a disease, disorder, syndrome, condition or symptom. Palliation of climacteric symptoms includes reducing the frequency and/or severity of hot flashes, insomnia, incontinence, depression, etc.

Daily Dose of Extract of Astragalus membranaceus, Astragalus mongholicus, Anemarrhenae asphodeloides and Achyranthes bidentata

The daily dose of a composition according to the invention (comprising an extract of Astragalus membranaceus, Astragalus mongholicus, Anemarrhenae asphodeloides and Achyranthes bidentata) for the treatment of climacteric symptoms, such as vaginal and/or vulvar atrophy will vary depending upon the age, weight, physical condition, and other attributes of the patient. In general, a daily dose of the composition comprising an extract of Astragalus membranaceus, Astragalus mongholicus, Anemarrhenae asphodeloides and Achyranthes bidentata will be from about 0.001 to about 100 grams dry weight of extract per day. In some embodiments, the dose will be about 1 to about 100 grams dry weight per day, about 1 to about 50 grams dry weight per day, about 10 to about 50 grams dry weight per day, about 20 to about 50 grams dry weight per day, about 10 grams dry weight per day, about 20 grams dry weight per day, about 30 grams dry weight per day, about 40 grams dry weight per day, about 50 grams dry weight per day, about 40 to about 100 grams dry weight per day, about 50 grams dry weight per day, about 60 grams dry weight per day, about 70 grams dry weight per day, about 80 grams dry weight per day, about 90 grams dry weight per day, or about 100 grams dry weight per day. Doses above 100 grams dry weight per day, e.g. about 100 to about 250 grams dry weight per day are also contemplated in some embodiments of the invention; however it is expected that suitable response will be obtained with a dose in the range of about 20 to about 80, especially about 30 to about 70 grams dry weight per day.

The daily dose may be administered in a single unit or may be administered in a divided daily dose comprising dosage units administered two, three, four or more times a day. A currently preferred dosage regimen is once-per-day (q.d.) or twice-per-day (b.i.d.) dosing, e.g. with the first dose being administered prior to, with, or after a morning meal and an optional second dose administered in mid-afternoon to bedtime, e.g. with an evening meal. In some embodiments, the daily dose is taken, either q.d. or b.i.d., as a tea, which optionally contains one or more flavorings, taste-masking agents and/or sweeteners.

The daily dose may be administered in other forms, especially other physical forms that are suitable for oral administration. In some embodiments, such other physical forms can include capsules. Other oral dosage forms suitable for administering compositions according to the invention include tablets (which optionally include one or more excipients, such as binders, disintegrants, diluents, glidants, or combinations of two or more thereof), caplets (which are understood to be a different shape from tablets but otherwise are similar in composition), chewable tablets, powders for solution or suspension, etc. Chewable tablets and powders for solution or suspension may contain one or more excipients, such as flavorings, taste-masking agents, sweeteners, and/or combinations of two or more thereof.

### EXAMPLES

The invention may be more fully appreciated with reference to the following illustrative and non-limiting examples.

### Example 1

ERβ is weaker than ERα at activating ERE-tkLuc: The effects of E₂ on transcriptional activation were examined by transfecting a plasmid containing a classical ERE upstream of the minimal thymidine kinase (tk) promoter linked to the luciferase reporter cDNA and an expression vector for ERα or ERβ. E₂ produced a 10-fold greater activation of the ERE in the presence of ERα compared to ERβ in human monocytic U937 cells, but the EC50 values were similar.

### Example 2

ERβ is more effective than ERα at repressing the TNF-RE-tkLuc: The effects of effects of E₂ on ERα and ERβ-mediated transcriptional repression were then compared using the −125 to −82 region of the TNF-α promoter, known as the tumor necrosis factor-response element (TNF-RE). TNF-α produced a 5-10-fold activation of 3 copies of the TNF-RE (-125 to -82) upstream of the tk promoter (TNF-RE tkLuc). E₂ repressed TNF-α activation of TNF-RE tkLuc by 60-80% in the presence of ERα and ERβ. However, ERβ was approximately 20 times more effective than ERα at repression (IC₅₀ of 241 pM for ERα versus 15 pM for and ERβ, respectively). It was also found that ERβ is more effective than ERα at repressing the native −1044 to +93 TNF-α promoter. Thus, ERα is much more effective than ERβ at transcriptional activation, whereas ERβ is more effective than ERα at transcriptional repression. In contrast to E₂, the antiestrogens, tamoxifen, raloxifene and ICI 182, 780 produced a 2-fold activation of TNF-RE tkLuc. Furthermore, these antiestrogens abolished the repression induced by E₂.

### Example 3

ERβ inhibits ERα-mediated transcriptional activation of ERE-tkLuc: Surprisingly, when ERα or ERβ were coexpressed in U937 cells, the activation by ERα were markedly inhibited. These data show that ERβ exerts a repressive effect on ERα activation of ERE-tkLuc. Similar results were observed in the breast cancer cell line, MDA-MB-435. Other investigators have found a similar repressive effect of ERβ on ERα transactivation in different cell types. These studies indicate that the different activation of ERα and ERβ on ERE-tkLuc and the repressive effect of ERβ on ERα-mediated-transcription are not cell-type specific and results from intrinsic properties of the ERs. The repression of ERα by ERβ requires the formation of an ERα/ERβ heterodimer, because mutations in helix 11 of ERβ that prevent dimerization inhibit its repression activity.

### Example 4

The drug of VG101 is a light brown to yellow oil of the ethyl acetate partition of an ethanol aqueous extract of three different plant species. It contains various ingredients of which three were determined to be ERβ selective agonists.

Nyasol, calycosin and tetracyclic isoflavone (TCIF) are the known active ingredients in VG101. Nyasol is isolated from the rhizome of Anemarrhenae asphodeloides Bunge (Liliaceae family); calycosin is isolated from the root of Astragalus membranaceus (Fisch.) Bunge and Astragalus mongholicus Bunge collectively as Radix Astragali, (Fabaceae/Leguminosae family); and TCIF is isolated from the root of Achyranthes bidentata B1. (Amarathaceae family).

TABLE
Composition of VG101

| | | Daily | % in Formula |
|---|---|---|---|
| | | Dose | Dose 1 |
| Herb Name [Family]¹ | Pin Yin² | (grams) | (w/w) |
| Rhizomae | Zhi Mu | 20 | 40 |
| Anemarrhenae | | | |
| asphoeloides [Liliaceae] | | | |
| Radix Astragali | Huang Qi | 20 | 40 |
| (Astragalus | | | |
| membranaceus Fisch. | | | |
| Bge. and Astragalus | | | |
| mongholicus Bge.) | | | |
| [Fabaceae/Leguminosae] | | | |
| Radix Achyranthes | Huai Niu Xi | 10 | 20 |
| bidentata Bl. | | | |
| [Amarathaceae] | | | |

| | | |
|---|---|---|
| Total | 50 grams | 100 |

¹Herb names are written in accordance with the international code for botanical nomenclature (Tokyo Code, 1994)
²In traditional Chinese medicine the designated Chinese name does not always designate one specific plant species. It is therefore important to define the specific species.

VG101 should be stored in a cool, dark, locked location.

### Example 5

When given by intra-vaginal administration to oopherectomized mice, at a dose of 0.5 mg/day for 10 days, VG101 resulted is non-keratinized squamous epithelium with normal maturation and glycogen rich vacuolated cells in the upper half of the epithelium. There was minimal, superficial chronic inflammation while estrogen treatment resulted in squamous epithelium with hyperkeratosis, characterized by a superficial layer of anucleated, keratinized squamous cells associated with a thickened granular layer.

In the mouse model, when compared to controls, VG101 resulted in more normal luteal phase endometrium with secretory glands and weakly decidualized stroma. VG101 administration was not associated with proliferation of endometrial glands while estrogen treatment resulted in endomyometrium (the equivalent of complex atypical hyperplasia in women, worrisome for in situ carcinoma). The glands form back to back without intervening stroma. They also showed numerous mitotic figures, markedly atypical nuclei with nucleoli. In one area, the glands arise in what appears to be a polyp with a broad stalk and thick fibrovascular core.

### Example 6

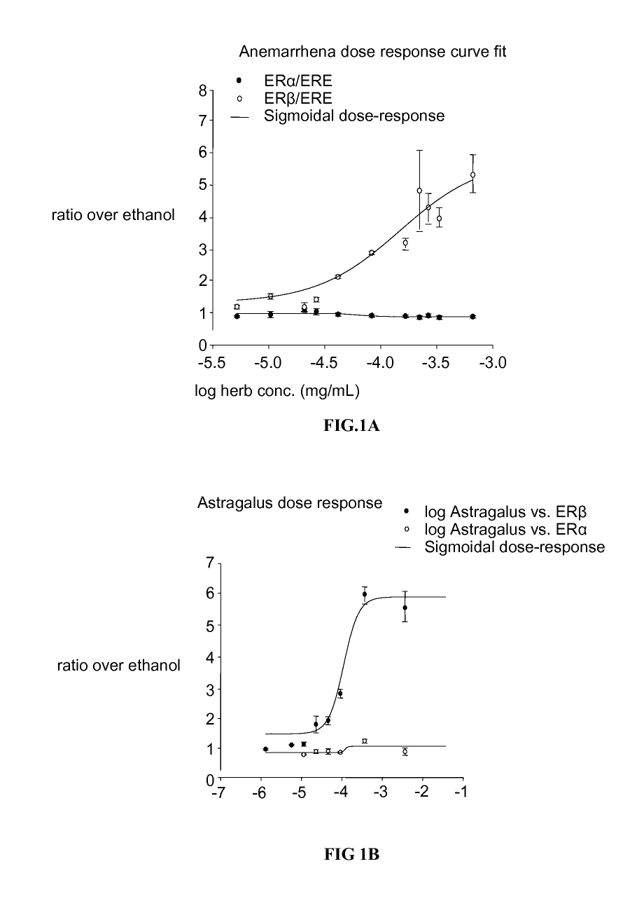

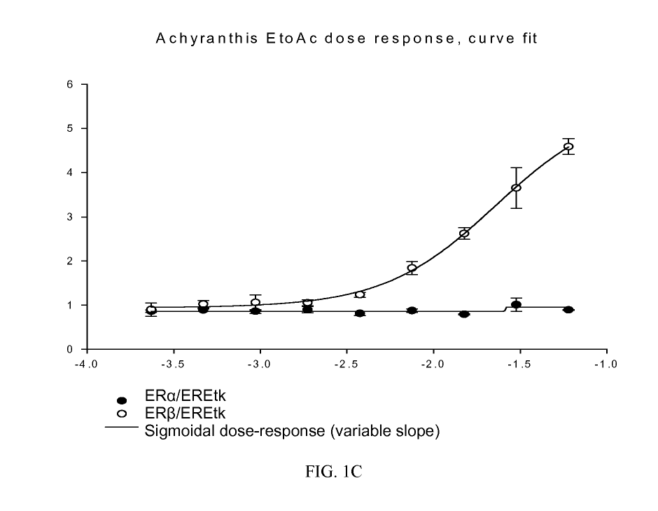

To determine if the herbal extracts and the active chemical compounds in VG101 have estrogenic activity, their effect on the regulation of an estrogen response element (ERE) upstream of a minimal thymidine kinase (tk) promoter (ERE-tk-Luc) was examined. U2OS osteosarcoma cells were co-transfected with ERE-tk-Luc and expression vectors for human ERα or ERβ. The extracts and their active chemical ingredients in VG101 produced a dose-dependent activation of ERE-tk-Luc with ERβ, but not with ERα (FIGS. 1A-1C). Amounts ranging from 1-125 μg/ml of herbal extracts and their active chemical ingredients in VG101 caused the activation equivalent to 10 nM estradiol (E₂) (FIGS. 1A-1C).

### Example 7

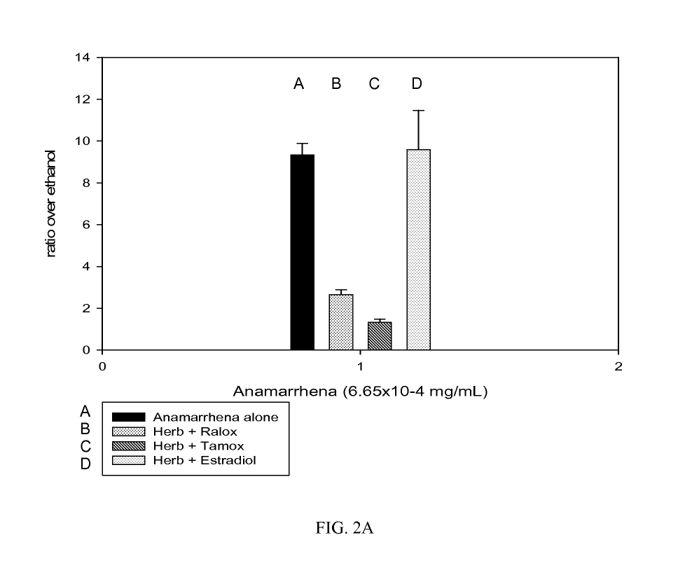

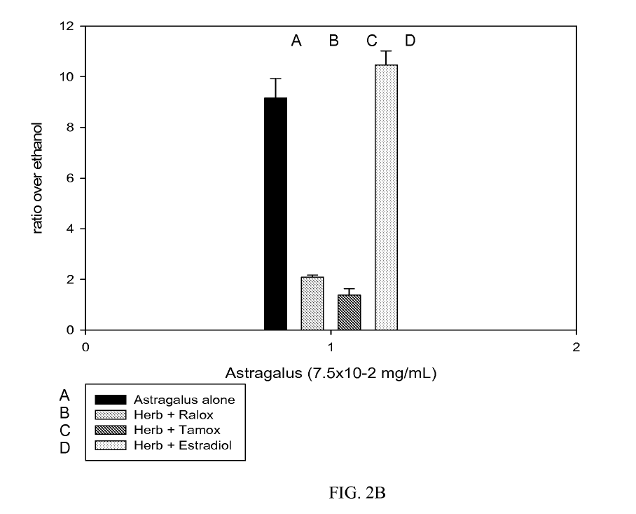

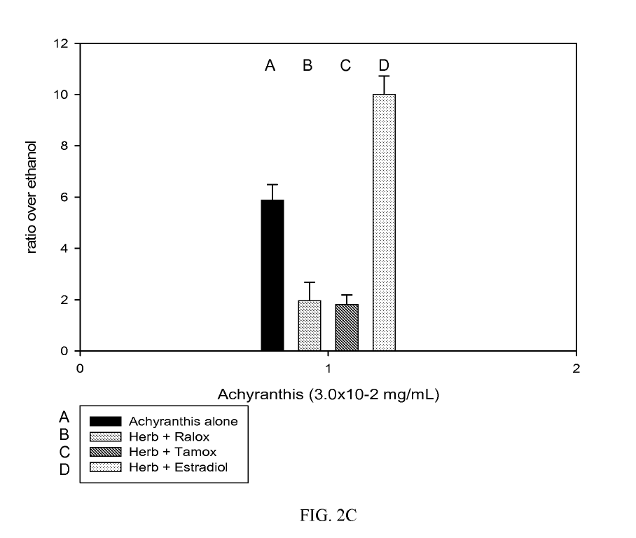

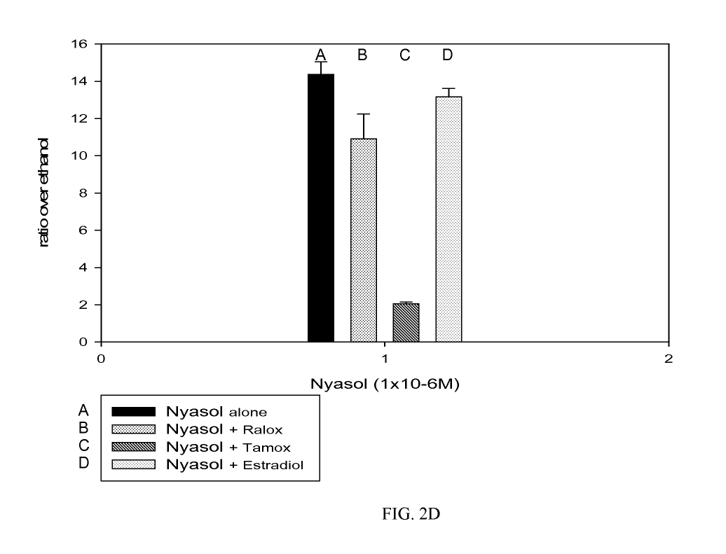

The ER antagonists ICI 182, 780, raloxifene (Ral) and tamoxifen (Tam) blocked the activation by herbal extracts and their active chemical ingredients in VG101 (FIGS. 2A-2D) indicating that the effect of VG101 is mediated through ERβ.

### Example 8

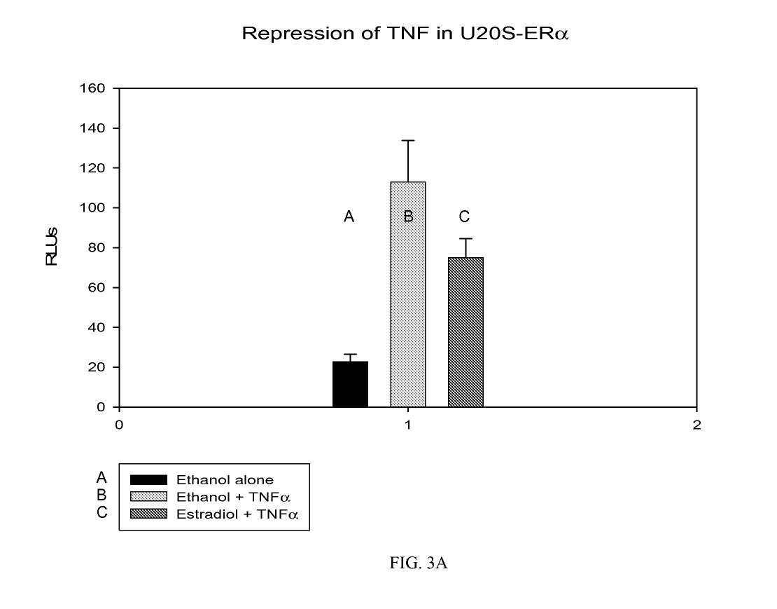

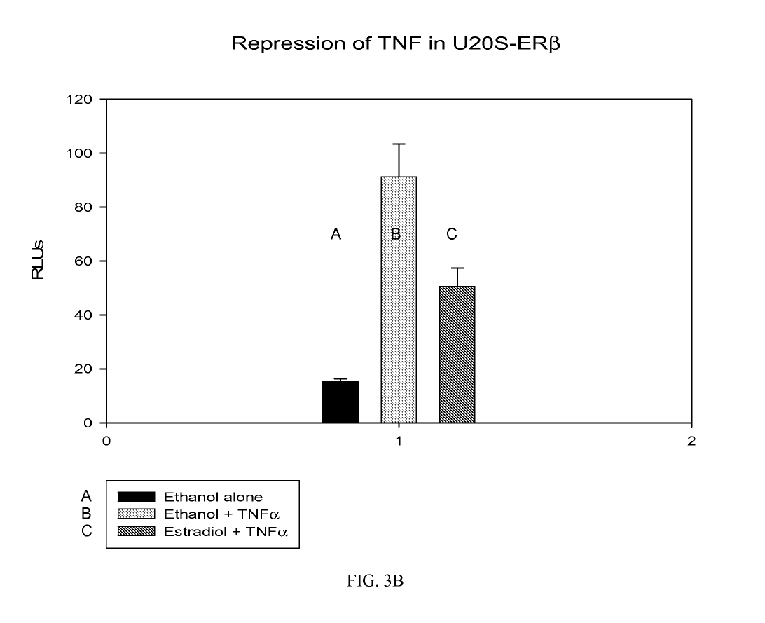

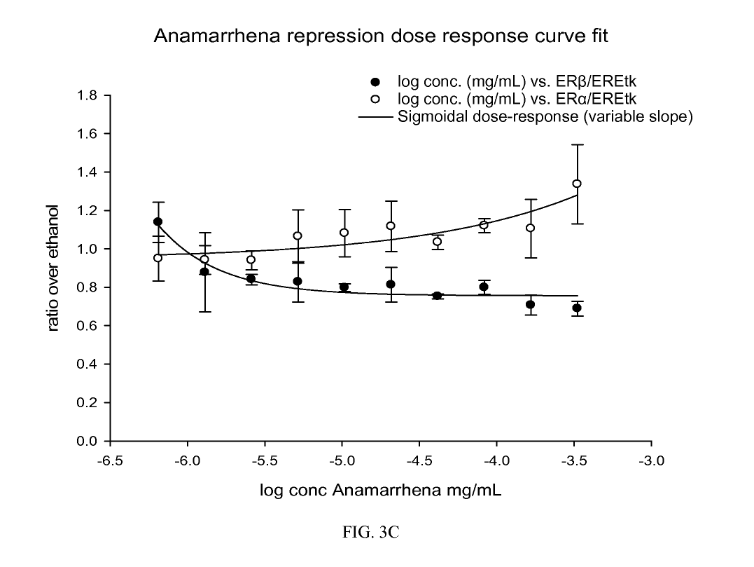

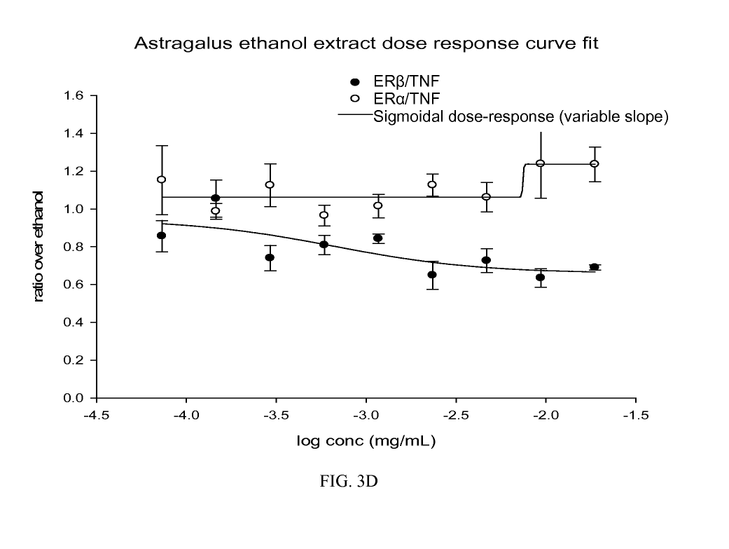

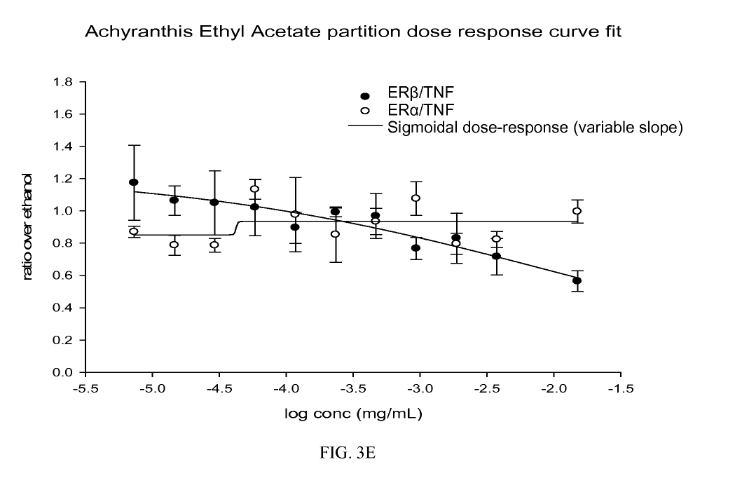

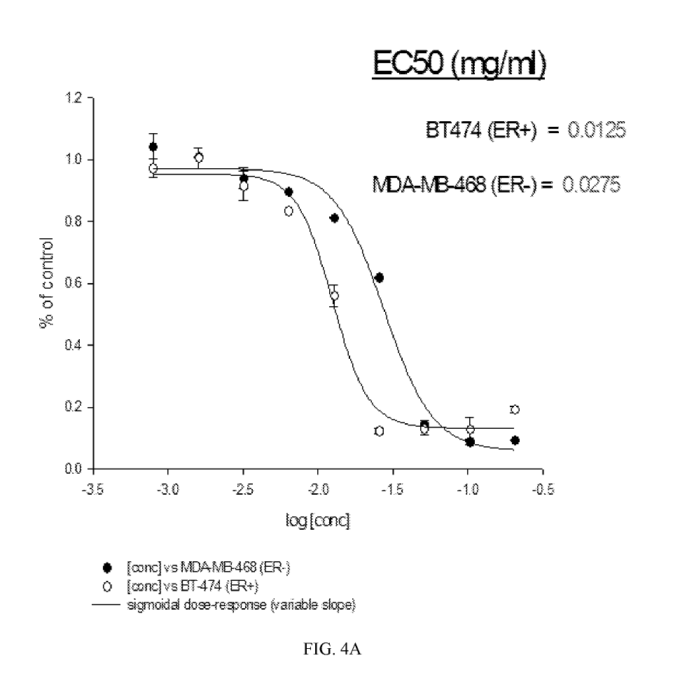

Estrogens possess anti-inflammatory properties by repressing the expression of inflammatory genes. The repression of the TNFα gene might be an important mechanism where estrogens prevent inflammatory conditions. To investigate if the herbal extracts and their active chemical ingredients in VG101 repress the expression of the TNFα gene, the tetracycline-inducible ERα or ERβ cells were treated with E₂ or herbal extracts and their active chemical ingredients in VG101. Because the basal expression of these genes is very low, it is necessary to activate these genes with TNFα to observe repression. TNFα produced a large increase in TNFα mRNA (FIGS. 3A-3B), which was inhibited by estradiol (E₂) in both the U2OS-ERα (FIG. 3A) and U2OS-ERβ (FIG. 3B). The herbal extracts and their active chemical ingredients in VG101 repressed the TNFα activation of the TNFα genes in the U2OS-ERβ cells (FIG. 3C-3E), but not in the U2OS-ERα cells (FIG. 3C-3E). These studies demonstrate that the herbal extracts and their active chemical ingredients in VG101 selectively trigger ERβ-mediated transcriptional pathways.

### Example 9

A critical feature of an alternative estrogen for menopausal symptoms is that it does not increase the risk for breast and uterine cancer. It was determined whether the herbal extracts and their active chemical ingredients in VG101 have growth promoting properties in breast cancer cells that express only ERα or express neither ERα nor ERβ.

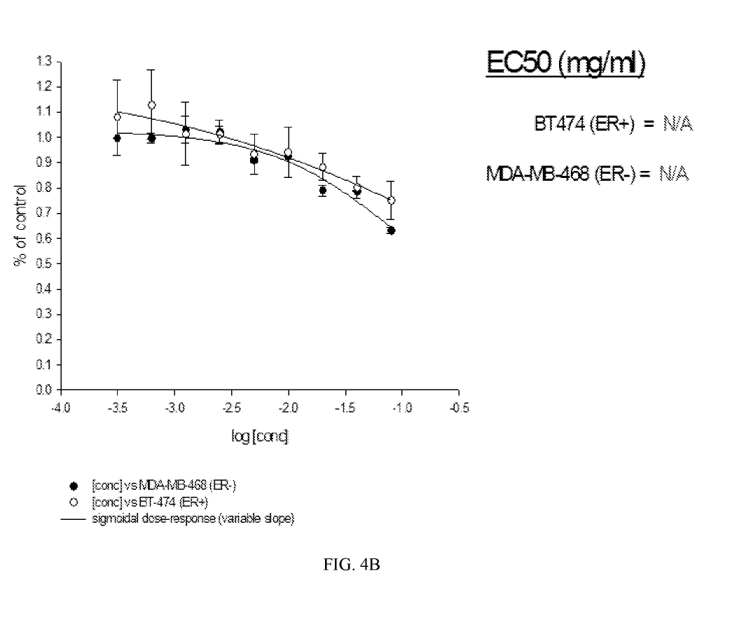

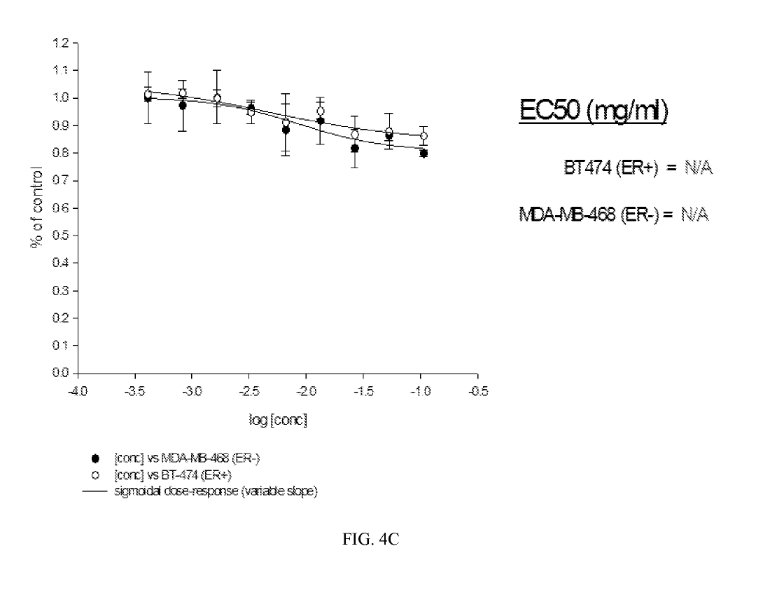

Proliferative effects are mediated by ERα and ERβ acts as a tumor suppressor that abolishes the proliferative effects of ERα. Based on these results, the herbal extracts and their active chemical ingredients in VG101 should not promote proliferation in breast cancer cells that express only ERα or express neither ER, because VG101 does not activate ERα or other growth promoting pathways in breast cancer cells. Unlike E₂, the herbal extracts and their active chemical ingredients in VG101 did not stimulate cell proliferation of BT474 cells or the cells that do not express ER, such as MDA-MB-468 (FIGS. 4A-4C).

### Example 10

In order to demonstrate efficacy of the Study Drug for the treatment of estrogenic disease states, the following double blind study is performed.

This study will include 4 cohorts of 10 healthy postmenopausal women (total N=40) in a prospective, randomized, blinded, placebo-controlled, dose escalation clinical trial evaluating the optimal dose of oral VG101 for the treatment of menopausal vaginal symptoms. Ten participants will be enrolled and randomized in a 4:1 ratio to the lowest dose of VG101 or to placebo and will be treated for up to 12 weeks. Study visits will take place at 4, 8 and 12 weeks.

After the cohort has completed 4 weeks of treatment, an independent Data and Safety Monitoring Board (DSMB) will examine participant data to evaluate toxicity of VG101 according to pre-defined safety and stopping rules. If no unacceptable toxicity is apparent, an additional 10 women will begin the study at the second higher dose and so on, until the primary outcome, optimal dose, is reached or until all four cohorts have completed 12 weeks of treatment.

The primary outcome measure will be unacceptable toxicity defined based on the National Cancer Institute (NCI) Common Toxicity Criteria for Adverse Events (CTCAE), version 3. Unacceptable toxicity is defined as the occurrence of a grade 3, 4 or 5 toxicity (NCI CTCAE version 3) that is possibly, probably or definitely related to study medication or any other unacceptable toxicity possibly, probably or definitely related to study medication, as determined by the DSMB.

Any adverse events will be recorded at each visit. As secondary outcomes, efficacy of VG101 will be measured by the change from baseline to 12 weeks of treatment in the vaginal symptoms (using a self-reported Likert scale), quality of life (using three validated self-reported questionnaires), physical examination findings (using a clinician-reported Likert scale), vaginal fluid pH, presence of lactobacilli and the proportion of parabasal vaginal epithelial cells. Adherence to study medication will be determined at each visit by applicator count and medication logs.

Study Population and Participant Eligibility

Participants will be approximately 40 postmenopausal women aged 45 to 65 years with moderate to severe menopausal vaginal symptoms who are willing to use an oral traditional Chinese herbal extract, specifically VG101.

Inclusion criteria:
Women between the ages of 45 and 65
Postmenopausal documented by: a) at least one year of amenorrhea; b) 6-12 months of amenorrhea with serum FSH >40 IU/ml; c) 6 weeks post-surgical bilateral oophorectomy with or without hysterectomy or d) hysterectomy with serum FSH levels >40 IU/ml.
Self-report at least one moderate to severe symptom of vulvar or vaginal atrophy from the following list:
   vaginal dryness (none, mild, moderate or severe)
   vaginal and/or vulvar discomfort (none, mild, moderate, severe)
   vaginal and/or vulvar irritation (none, mild, moderate, severe)
   vaginal itching (none, mild, moderate, severe)
   vaginal pain associated with sexual activity (none, mild, moderate or severe)
   ≦5% superficial cells on vaginal cytologic smear.
   Vaginal pH >5.0.
Willing to use an oral traditional Chinese herbal medicine extract for the treatment of vaginal symptoms.
Currently receive medical care from a health care provider.
Provide informed consent.

Exclusion criteria
History of breast, uterine or ovarian cancer or melanoma.
Abnormal mammogram or breast examination within the last 9 months suggestive of cancer.
Abnormal Pap smear or pelvic examination within the last 9 months suggestive of cancer.
Any uterine or vaginal bleeding within the six months prior to enrollment (except following the screening Pap smear).
Double-wall endometrial thickness that exceeds 5 mm measured on transvaginal ultrasound.
Pregnant or lactating.
Use of any vaginal moisturizer (Replens, KY Silk-E, Astroglide Silken Secret, Senselle) within 30 days of screening.
Use of any oral, transdermal, vaginal or systemic estrogen or estrogen/progestin product within 30 days of screening.
Use of prescription medications known to possibly be effective for the treatment of hot flashes within three months of enrollment for oral or transdermal drugs or within 6 months of enrollment for implanted or injected drugs.
Use of raloxifene, tamoxifen or aromatase inhibitors within 12 weeks of screening.
Current urinary tract infection (dipstick urinalysis positive for leukocyte estrace, nitrites or blood)
Clinical evidence of active ischemic cardiovascular disease or history of cardiovascular disease.
History of deep vein thrombosis or pulmonary embolism requiring anticoagulation.
History of stroke.
Active liver or gallbladder disease.
History of severe medicine allergies.
Use of another investigational agent within 12 weeks of screening.
Any medical or psychiatric condition that, in the investigator's opinion, would preclude the participant from completing questionnaires or measures, adhering to the protocol or completing the trial, including limited English literacy, severe illness, plans to move, substance abuse, significant psychiatric problems or dementia.
No access to a telephone.

Informed Consent and Documentation

Before entering the study, all study procedures, time requirements, risks and potential benefits will be explained to each potential study participant using the information in the informed consent form. The potential study participant will be given adequate time to read the informed consent document and ask questions. Eligible participants who choose to enter the study will sign an IRB-approved consent prior to beginning study treatment. Each participant will be given a copy of the signed consent and the original will be a part of the research record. All study-specific data will be confidential and held in locked files at the clinical center.

Study Drug

Oral VG101 will consist of a combination of three herbal compounds packaged in gel capsules. Oral VG101 will be self-administered twice a day for 12 weeks. The formulation was derived from the three Chinese herbs Anemarrhena asphodeloides Bunge (Pin Yin: Zhi Mu), Astragalus membranaceus Fisch. Bge. Var. mongholicus Bge. (Pin Yin: Huang Qi) and Achyranthes bidentata B1. (Pin Yin: Huai Niu Xi) and will have an FDA approved Investigational New Drug (IND) license prior to the commencement of the trial.

Standardization of the raw materials will be controlled for by precise botanical species identification by a traditional Chinese medicine expert. A secondary pharmaceutical assessment will include extracting each of the three herbs in accordance with the FDA IND specifications. All botanical agents will be identified against a taxonomic marker via thin layer chromatography, LC/MS/MS analytical quantification of the active components, using Applied Biosystems API5000. The taxonomic marker identifying band of the plant extract will be placed on a silica gel, using ethyl acetate as an eluent to ensure that the herbal extract matches the pure compound with >90% accuracy. The LC/MS fingerprint of the extract will be matched between batches to ensure the components of the extract are consistent.

VG101 will be prepared with the above ingredients using pure compounds and FDA Good Manufacturing Procedures. The study medication will be packaged in plastic bottles and labeled according to FDA guidelines.

The matching placebo will be made with the inactive ingredient and matched for color to the active drug.

Administration of the Drug and Dosing Schedule

The study drug will be administered orally, twice a day, for 12 weeks. Participants will be instructed to take one gel capsule in the morning and one at night for the entire 12 week study beginning the evening of the Randomization Visit.

The first 10 eligible participants will be randomized (in a 4:1 randomization ratio) and given oral VG101 at a starting dose of 25 mg PO BID of VG101 or identical placebo. Recruitment and randomization will be continuous, with the second cohort of 10 eligible participants assigned to a dose of 125 mg PO BID, the third cohort to 250 mg PO BID and the final cohort to 500 mg PO BID. All participants will be asked to continue on the assigned dose for 12 weeks unless unacceptable toxicity is observed

Dose Escalation Schedule

| Dose Level | Dose of VG101 |
|---|---|
| Level 1 | 25 mg PO BID |
| Level 2 | 125 mg PO BID |
| Level 3 | 250 mg PO BID |
| Level 4 | 500 mg PO BID |

Unacceptable Toxicity and Optimal Dose

Unacceptable toxicity is defined as the occurrence of a grade 3, 4 or 5 toxicity using the NCI Common Terminology Criteria for Adverse Events (CTCAE), version 3 that is possibly, probably or definitely related to study medication that is possibly, probably or definitely related to study medication or any other unacceptable toxicity identified by the Data and Safety Monitoring Board that is possibly, probably or definitely related to study medication.

A dose level will be terminated if: 1) unacceptable toxicity is observed among 1 or more of 8 women assigned to active treatment in any cohort at any time during 12 weeks of treatment. If a dose level is terminated, the next lowest dose will be designated the optimal dose. If unacceptable toxicity is not observed in any cohort during 12 weeks of treatment, the highest dose 500 mg PO BID of VG101 will be designated the optimal dose.

Adverse Events

Adverse events will be classified by general organ system and graded for severity (1-5) according to the NCI-CTCAE, version 3. Adverse events will be ascertained at each visit and reported by clinical site investigators. Detailed information will be collected for each adverse event including a description of the event, duration, whether the adverse event was serious, the nature of the event (single episode vs. multiple episode), intensity, relationship to study drug, action taken, clinical outcome and whether the adverse event resulted in surgery or an alternate procedure.

In addition to classification by NCI-CTCAE, adverse events will be classified using MedDRA software to preferred terms and body systems. Adverse events will also be classified by severity, relationship to study drug and whether the adverse event resulted in discontinuation of study medication. Baseline laboratory or medical conditions that worsen to grade III or above do not constitute a dose limiting toxicity unless they are coded as possibly, probably or definitely associated with VG101 therapy.

Visit Schedule

Initial Screening Evaluation (Telephone)

Women who respond to study advertisements and notices will be provided with a general overview of the study and, if interested, will complete a brief phone survey to determine initial eligibility (age, menopausal status, medication use, symptoms of vaginal dryness, date of last mammogram and willingness to participate in randomized trial). Eligible respondents will be invited to attend a screening visit to determine eligibility. Participants will be asked to bring all medications, including prescription and over-the-counter preparations, to the visit.

Screening Visit

Participants will be asked not to have sexual intercourse, nor to douche or use vaginal lubricants, etc. for at least 12 hours prior to coming to the clinic. They may take any usual medications. At the Screening Visit(s), women will be informed about the details and requirements of the study and will provide informed consent. Blood will be drawn and urine collected for safety and pharmacokinetic studies. Participants will complete questionnaires covering inclusion and exclusion criteria, demographics, brief medical, surgical, gynecological and menstrual history and symptoms of vulvar and vaginal atrophy. Current medications will be reviewed.

A brief physical exam, including blood pressure, height, weight, breast and pelvic examination will be completed. Vaginal atrophy will be assessed using the Vaginal Health scale to assess vaginal atrophy and inflammation. A Pap smear and cellular samplings of the mid lateral vaginal wall for cervical and vaginal cytology will be performed to determine vaginal epithelial cell maturation and the presence of vaginal estrogen receptors. Vaginal pH will be measured and a small sample of vaginal fluid will be obtained for microbial analysis. Quantification of pelvic organ prolapse (POP-Q) will be performed. Study staff will request permission to obtain results of any mammogram performed within the last nine months. If the participant has not had a mammogram within 9 months, she will be scheduled to have one prior to being randomized. A screening transvaginal ultrasound will be scheduled and the results will be reviewed prior to randomization. The participant will be given a Screening Daily Bleeding Diary with instructions for filling it out. She will be asked to bring the completed diary to the next visit.

Randomization Visit

The clinical site investigator will review the results of all measurements prior to the randomization visit. Any bleeding documented on the bleeding diary that is not attributable to the performance of the Pap smear will render the participant ineligible. Participants will be considered eligible to continue if they meet all inclusion and exclusion criteria. Eligible participants will receive verbal and written instructions describing dosing of the study medication and potential side effects. Participants will complete the Female Sexual Function Index, the Menopause Specific Quality of Life, SF36 and Pelvic Floor Impact questionnaires and the Pelvic Floor Distress Inventory. A randomization number will be assigned sequentially and a 4 week supply of study medication will be dispensed. Participants will be given a Daily Bleeding Diary and asked to return the completed diaries to the next visit.

All women who enroll in the trial will be given information on how to contact study personnel. An appointment will be made for the 4 week visit.

Two Week and Eight Week Visits (Telephone Visits)

At 2 and 8 weeks of treatment, the coordinator will call the study participant to review medication compliance, adverse effects, concurrent medications, the Daily Study Medication Diary and the Daily Bleeding Diary and to answer any questions. These visits will be scheduled as a phone visit; however, at the participant's request, these visits may also be conducted in person.

Four Week Visit

After four weeks of treatment, the participant will return to the clinic bringing with her any unused study medication and the Daily Study Medication Diary and the Daily Bleeding Diary. The study coordinator will count the unused study medication and calculate medication compliance. The following will be reviewed by the study coordinator: adverse effects and new concurrent medications will be recorded and the Daily Study Medication Diary and the Daily Bleeding Diary. Participants will complete the same quality of life questionnaires that they completed at baseline. Another 8 weeks of study medication will be dispensed.

Twelve Week Final Visit or Study Termination

A final visit will take place after 12 weeks of treatment or at any time that a participant decides to discontinue participation in the trial. At Final Visit, participants will bring in their Daily Study Medication Diary and the Daily Bleeding Diary. Blood will be drawn and urine collected for safety studies. All questionnaires, physical examination, laboratory analyses and vaginal studies completed at screening will be repeated at study completion. A second transvaginal ultrasound will be performed and any study participant with a double-wall endometrial thickness >5 mm or endometrial wall thickness that has increased more than 2 mm from screening will have an endometrial aspiration biopsy. All unused study medication will be collected at this time.

Sixteen Week Off-Study Follow Up Telephone Visit or Four Weeks Post Study Termination

A Follow Up Telephone Visit will take place at 16 weeks or 4 weeks after the participant has discontinued participation in the trial. The participant will be contacted by the study coordinator to ascertain any new adverse effects since the final visit and to review any ongoing adverse effects.

Non-Adherence Study Drug Discontinuation and Early Study Discontinuation

All participants will be encouraged to use all doses of study medication as instructed unless safety is a concern. Study coordinators will ascertain reasons for non-adherence to study medication and attempt to help participants find ways to improve adherence. Participants who are not adherent with study medication will be urged to attend all study visits and complete all study measurements as planned. Adherence with study medication will be measured by applicator counts at each visit and checked with the Study Medication Calendar.

If a study participant reports vaginal bleeding, she may continue on study medication, but must undergo evaluation for the bleeding within 4 weeks. If no evaluation occurs within 4 weeks, the participant must stop study medication until the evaluation is complete.

Study drug will be discontinued in any participant who has: 1) endometrial hyperplasia or cancer on endometrial biopsy; 2) abnormal pelvic examination suggestive of uterine or ovarian cancer; 3) breast examination or mammogram suggestive of cancer; 4) deep vein thrombosis; 5) diagnosis of dementia and 6) any serious adverse event that, in the judgment of the clinical investigator, is possibly, probably or definitely related to treatment with VGF101. Study medication may be resumed if, in the opinion of the clinical site investigator, the abnormal symptom, physical finding or test has been satisfactorily evaluated and found to be benign.

Participants will be considered to have permanently discontinued study medication if they report not taking study medication for 4 weeks or longer or if they state that they no longer desire to participate in the trial. In this case, study termination measurements will be obtained if possible.

All participants will be urged to attend all study visits and complete all study measurements as planned. However, participants may discontinue participation in the study at any time. Participants who miss a visit will be contacted by the study coordinator to reschedule the visit and to provide assistance in completing the visit.

Following enrollment, participants should begin protocol treatment within 72 hours. Issues that would cause treatment delays should be discussed with the principal investigator. If a participant does not start protocol therapy following enrollment, the participant's enrollment in the study may be canceled.

Early termination of the study for unanticipated benefit or harm may be recommended to Bionovo and the Steering Committee by the Data and Safety Monitor.

Outcomes Measures

### Primary Vaginal Atrophy Measures

Change in self-reported, most bothersome symptom and change in symptoms of vaginal atrophy from baseline to 12 weeks. Symptoms of vaginal atrophy will be assessed by self-administered questionnaire at baseline, 4 weeks and at 12 weeks or study termination. The questionnaire will consist of 7 items assessing vaginal dryness, vaginal irritation, vaginal itching, vaginal pain with sexual activity, vaginal bleeding with sexual activity, pain or discomfort when urinating (dysuria) and frequent need to urinate (urinary frequency). Participants will be asked if they had the symptom in the past month (yes=1, no=0) and, if so, how much the symptom bothered them, with response options including “not at all” (0), “a little bit” (1), “moderately” (2), “quite a bit” (3) or “extremely” (4). At baseline, participants will identify the most bothersome symptom of vaginal atrophy. Baseline scores (range 1-4) for the most bothersome symptom of vaginal dryness will be compared to the score for the same symptom at week 12. In addition, composite scores for symptoms of vaginal dryness (range=0-35) at baseline will be compared to week 12.

Change in percent superficial vaginal epithelial cells from baseline 12 weeks. Diagnostic Cytology Laboratories, Inc. will provide data relating to the maturation of the squamous epithelium of the vaginal mucosa by microscopic evaluation of vaginal epithelial cells. Three basic patterns of vaginal maturation can be distinguished (immature, intermediate and mature) based on the predominance of different cell types:

| Pattern | Immature | Intermediate | Mature |
|---|---|---|---|
| Cell Type | Parabasal (P) | Intermediate (I) | Superficial (S) |
| Maturation | 100/0/0 (P/I/S) | 0/100/0 (P/I/S) | /0/0/100 (P/I/S) |
| Index Value | | | |

A sample will be taken from the mid to upper ⅓ of the lateral wall of the vagina, the epithelium most sensitive to hormonal changes, during vaginal examination. Satisfactory specimens for hormonal evaluation will be free from inflammation, bacteria or other debris and will have been mounted and fixed according to laboratory instructions. Among the satisfactory specimens, 200 single cells will be counted and the relative percentages of parabasal, intermediate and superficial cells will be noted. Samples considered invalid (e.g. the sample contains inflammatory cells) will be submitted for additional interpretation by a supervisory cytotechnologist and in some instances, these cases may require review by a pathologist.

After counting 200 squamous cells, a numeric Maturation Value, which is a rough estimate of estrogenicity, will be generated by assigning a point value to each cell type (parabasal=0.0, intermediate=0.5 and superficial=1.0) and multiplying each cell type count by its point value. The results will be expressed as low estrogenicity (0-49), moderate estrogenicity (50-64) and high estrogenicity (65-100). Additionally the percent of superficial cells at week 12 will be compared to the percent of superficial cells at baseline.

Change in the pH of vaginal secretions from baseline to 12 weeks. The pH of vaginal secretions will be recorded at baseline and compared to the vaginal pH at study termination. A cotton-tip applicator will be used to take a sample of the vaginal fluid from the mid-lateral vaginal wall; the tip of the applicator will then be pressed against pH indicator strips capable of distinguishing between 0.3-unit differences in pH from a range of 2.5 to 7.0. Previous studies have suggested that pH indicator strips provide accurate measurements of vaginal pH in comparison to pH micro electrode.

Change in physical signs of vaginal atrophy on physical exam. The physician's assessment of the vaginal wall will include assessment of elasticity, rugosity, moisture, petechiae and friability. Abnormality of findings in each of these categories will be rated as absent=0, mild=1 or severe=2. Composite scores for the physical examination scale will range from 0 to 10. The percent of participants who change one or more categories will be compared from baseline to week 12. Also, composite scores at baseline will be compared to composite scores at study termination. Additionally, physical exam data will be used to calculate a modified Greendale vaginal atrophy score, a previously validated physical examination instrument that notes the presence or absence of petechiae, vaginal wall friability, conization and rugae and assigns a score of 0 to 4, with higher scores indicating more severe atrophy.

Change in Vaginal Microbial Flora. Vaginal fluid specimens from the lateral vaginal wall will be sent in transport medium for microbial examination, which may include Nugent scoring of the gram-stain smear as well as quantitative culturing of Lactobacilli, E. Coli and non-E. Coli gram-negative rods. Changes from baseline to week 12 in the types and quantities of organisms present will be determined.

### Secondary and Quality-of-Life Measures:

The Female Sexual Function Index (FSFI) is a validated, 19-item, self-reported questionnaire that assesses sexual function in women. This index was designed for use in clinical trials and has been shown to have good test-retest reliability and internal consistency in both community-based and referral populations. Scoring algorithms are available for both the overall instrument and for specific domains of sexual function such as desire, arousal, lubrication orgasm, satisfaction and pain. Composite scores range from 2.0 to 36.0, with higher scores indicating better overall sexual function. The percent change in composite score and in each domain score from baseline to 4 and 12 weeks of treatment will then be determined.

The Menopause-specific Quality of Life (MENQOL) is a self-administered, 30-item questionnaire with four domains, including vasomotor, physical, psychosocial and sexual function. Each domain is scored separately on a one to eight point scale. Adequate test-retest reliability, good responsiveness and good to excellent discriminative and evaluative construct validity have been demonstrated. Percent change in each domain of the MENQOL from baseline to 4 and to 12 weeks of treatment will then be determined.

Menopausal Symptom Questionnaire—a brief Menopausal Symptom Questionnaire ascertains information on the bothersomeness of typical symptoms experienced by menopausal women such at hot flashes, breast tenderness, vaginal dryness and discharge and trouble sleeping. This questionnaire has been used and validated in multiple major trials including the Postmenopausal Estrogen-Progestin Interventions trial the Heart and Estrogen/progestin Replacement Study and the Women's Health Initiative trials. Each symptom is rated on a 5-point Likert scale ranked 0 (no bothersomeness) to 4 (very bothersome). The percent change in each of these scores from baseline to 4 and to 12 weeks of treatment will then be assessed.

SF-36—is a short form, nonspecific health survey that was designed to measure health constructs that are most affected by disease and treatment. The SF-36 is a validated instrument that has been widely used to compare the relative burden of disease in populations and the health benefits associated with treatment. The SF-36 has a physical and mental health subscale. The percent change in each subscale (physical and mental health) from baseline to 4 and to 12 weeks of treatment will then be measured.

Pelvic Organ Prolapse Quantitation (POP-Q)—The pelvic organ prolapse evaluation will be performed according to the guidelines established by the International Continence Society using the Pelvic Organ Prolapse Quantification (POP-Q) system. The procedure will be standardized as demonstrated in a videotape produced by Duke University Medical Center.

Abbreviated versions of two condition-specific quality-of-life questionnaires for women with pelvic floor disorders, the Pelvic Floor Distress Inventory (PFDI)-20 and the Pelvic Floor Impact Questionnaire(PFIQ)-7 were developed by Barber et al from data from the 100 women who contributed to the development and validation of the Pelvic Floor Distress Inventory and Pelvic Floor Impact Questionnaire long forms.

Pelvic Floor Distress Inventory—The PFDI assesses symptom distress in women with pelvic floor disorders on 3 scales: the Urogenital Distress Inventory, Colorectal-anal Distress Inventory and Pelvic Organ Distress Inventory.

Pelvic Floor Impact Questionnaire—short form 7—The PFIQ assesses life impact on 3 scales: Incontinence Impact Questionnaire, Colorectal-anal Impact Questionnaire and the Pelvic Organ Prolapse Impact Questionnaire.

### Coverables

The following data will be collected on all participants at baseline:
Demographics: Date of birth, race, ethnicity, relationship status, education.
Medical and Surgical History: Gynecological surgeries (number of ovaries removed), breast cancer, ovarian cancer, tobacco use and alcohol use.
Reproductive and Menstrual History: Gravidity, parity, last menstrual period, age of menarche.
Concurrent Medications: Medications taken currently or during past three months, including over-the-counter medications and dietary supplements.
History of Menopausal Symptoms: age of onset of menopausal symptoms, past treatment for menopausal symptoms.

While preferred embodiments of the present invention have been shown and described herein, it will be obvious to those skilled in the art that such embodiments are provided by way of example only. Numerous variations, changes and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition, comprising a therapeutically effective amount of an extract of Astragalus membranaceus, Astragalus mongholicus, Anemarrhenae asphodeloides and Achyranthes bidentata, wherein the extract comprises at least one phytochemical.

2. The composition of claim 1, further comprising one or more pharmaceutically acceptable excipients.

3. The composition of claim 1, wherein the phytochemical is selected from the group consisting of: calycosin, nyasol and tetracyclic isoflavone.

4. The composition of claim 3, wherein the extract comprises at least two of: calycosin, nyasol and tetracyclic isoflavone.

5. The composition of claim 3, wherein the extract comprises calycosin, nyasol and tetracyclic isoflavone.

6. The composition of claim 1, wherein the purified extract of phytochemicals is obtained by performing a first extraction and a second extraction.

7. The composition of claim 1, wherein the composition further comprises at least one additional ingredient selected from the group consisting of: active pharmaceutical ingredients; enhancers; excipients; and agents used to adjust the pH, buffer the composition, prevent degradation and improve appearance, odor or taste.

8. A process for isolating a purified extract of phytochemicals from the plant species Astragalus membranaceus, Astragalus mongholicus, Anemarrhenae asphodeloides and Achyranthes bidentata, comprising a first extraction and a second extraction, wherein the first extraction comprises the steps of:
(a) dispersing Astragalus membranaceus, Astragalus mongholicus, Anemarrhenae asphodeloides and Achyranthes bidentata in a first solvent to form a first mixture;
(b) heating the first mixture; and removing the first solvent to obtain a first extract; and the second extraction comprises the steps of:
(a) mixing the first extract with a second solvent to produce a second mixture;
(b) allowing the second mixture to partition into an aqueous layer and a non-aqueous layer;
(c) collecting the non-aqueous layer;
(d) removing any non-active compounds; and
(e) removing the second solvent to obtain a purified extract of phytochemicals.

9. The process of claim 8, wherein the first solvent is a solution of ethanol and water.

10. The process of claim 8, wherein the first mixture is heated to a temperature from about 10° C. to about 60° C.

11. The process of claim 8, wherein the first mixture is heated for about 2 hours to about 6 hours.

12. The process of claim 8, wherein the first solvent is removed from the first extract by evaporation.

13. The process of claim 17, wherein the evaporation takes place at a temperature from about 40° C. to about 60° C.

14. The process of claim 8, wherein the phytochemical is selected from the group consisting of: calycosin, nyasol and tetracyclic isoflavone.

15. The process of claim 8, wherein the extract comprises at least two of: calycosin, nyasol and tetracyclic isoflavone.

16. The process of claim 8, wherein the extract comprises calycosin, nyasol and tetracyclic isoflavone.

17. A method of treating a patient that has a disorder, comprising administering to the patient a composition comprising a therapeutically effective amount of a purified extract of phytochemicals from Astragalus membranaceus, Astragalus mongholicus, Anemarrhenae asphodeloides and Achyranthes bidentata.

18. The method of claim 17, wherein the phytochemical is selected from the group consisting of: calycosin, nyasol and tetracyclic isoflavone.

19. The method of claim 17, wherein the extract comprises at least two of: calycosin, nyasol and tetracyclic isoflavone.

20. The method of claim 17, wherein the extract comprises calycosin, nyasol and tetracyclic isoflavone.

21. The method of claim 17, wherein the composition further comprises at least one additional ingredient selected from the group consisting of: active pharmaceutical ingredients; enhancers; excipients; and agents used to adjust the pH, buffer the composition, prevent degradation and improve appearance, odor or taste.

22. The method of claim 17, wherein the composition is administered by a dosage form selected from the group consisting of: as a solid oral dosage form, liquid oral dosage form, gelatin capsule dosage form, a vaginal suppository, a rectal suppository or a spray.

23. The method of claim 17, wherein the therapeutically effective amount is an amount which elicits an estrogenic effect.

24. The method of claim 23, wherein the estrogenic effect is selected from the group consisting of: treating or preventing at least one climacteric symptom; treating or preventing osteoporosis; or any combination thereof.

25. The method of claim 24, wherein the estrogenic effect is treating or preventing at least one climacteric symptom.

26. The method of claim 25, wherein the climacteric symptom is selected from the group consisting of: hot flashes, insomnia, vaginal or vulvar atrophy, decreased libido, urinary incontinence, headache, depression or any combination thereof.

27. The method of claim 26, wherein the climacteric symptom is vaginal or vulvar atrophy.

28. The method of claim 23, wherein the estrogenic effect includes treating or preventing osteoporosis.

29. A method of activating a gene under control of an estrogen response element, comprising administering to a cell having an estrogen response element operatively linked to the gene and an estrogen receptor an amount of a composition, wherein the composition comprises a purified extract of phytochemicals from Astragalus membranaceus, Astragalus mongholicus, Anemarrhenae asphodeloides and Achyranthes bidentata, and wherein the purified extract of phytochemicals is in an amount sufficient to activate the gene.

30. A method of repressing expression of a TNF RE-controlled gene, comprising administering to a cell comprising a gene under control of a TNF response element and an estrogen receptor an amount of a composition, wherein the composition comprises a purified extract of phytochemicals from Astragalus membranaceus, Astragalus mongholicus, Anemarrhenae asphodeloides and Achyranthes bidentata and wherein the purified extract of phytochemicals is in an amount sufficient to repress the TNF RE-controlled gene.
